(19) United States
(12) Patent Application Publication
(10) Pub. No.: US 2014/0309400 A1
(43) Pub. Date: Oct. 16, 2014

(54) Use of Cell-Permeable Peptide Inhibitors of the JNK Signal Transduction Pathway for the Treatment of Dry Eye Syndrome

(71) Applicant: Xigen Inflammation Ltd., Limassol (CY)

(72) Inventors: Jean-Marc Combette, Saint Cergues (FR); Catherine Deloche, Genf (CH)

(21) Appl. No.: 14/361,529

(22) PCT Filed: Nov. 30, 2012
(86) PCT No.: PCT/EP2012/004952

§ 371 Date: May 29, 2014

(30) Foreign Application Priority Data
Nov. 30, 2011 (EP) PCTEP2011006003

(51) Int. Cl. C07K 14/47

(52) U.S. Cl. CPC C07K 14/47
USPC 530/324; 536/23.5; 435/320.1; 435/325; 435/348; 435/254.2; 435/252.3

(57) ABSTRACT

The present invention refers to the use of protein kinase inhibitors and more specifically to the use of inhibitors of the protein kinase c-Jun amino terminal kinase, JNK inhibitor (poly-)peptides, chimeric peptides, or of nucleic acids encoding same as well as pharmaceutical compositions containing same, for the treatment of dry eye syndrome.

The present invention refers to the use of protein kinase inhibitors and more specifically to the use of inhibitors of the protein kinase c-Jun amino terminal kinase, JNK inhibitor (poly-)peptides, chimeric peptides, or of nucleic acids encoding same as well as pharmaceutical compositions containing same, for the treatment of dry eye syndrome.

Dry eye syndrome (DES), also called keratitis sicca, xerophthalmia, keratoconjunctivitis sicca (KCS) or cornea sicca, is an eye disease caused by eye dryness, which, in turn, is caused by either decreased tear production or increased tear film evaporation. Typical symptoms of dry eye syndrome are dryness, burning and a sandy-gritty eye irritation. Dry eye syndrome is often associated with ocular surface inflammation. If dry eye syndrome is left untreated or becomes severe, it can produce complications that can cause eye damage, resulting in impaired vision or even in the loss of vision. Untreated dry eye syndrome can in particular lead to pathological cases in the eye epithelium, squamous metaplasia, loss of goblet cells, thickening of the corneal surface, corneal erosion, punctate keratopathy, epithelial defects, corneal ulceration, corneal neovascularization, corneal scarring, corneal thinning, and even corneal perforation.

The object of the present invention is thus to provide a medicament, which allows treatment of dry eye syndrome and/or associated pathological effects, symptoms, etc.

This object is solved by the use of a JNK inhibitor (poly-)peptide comprising less than 150 amino acids in length for the preparation of a pharmaceutical composition for treating dry eye syndrome in a subject.

The present inventors surprisingly found, that JNK inhibitor (poly-)peptides are particularly suitable for treating dry eye syndrome in a subject. This was neither obvious nor suggested by the prior art, even though JNK inhibitor (poly-)peptides in general have been known from the art.

In the context of the present invention, a JNK inhibitor (poly-)peptide may be typically derived from a human or rat IB1 sequence, preferably from an amino acid sequence as defined or encoded by any of sequences according to SEQ ID NO: 102 (depicts the IB1 cDNA sequence from rat and its predicted amino acid sequence), SEQ ID NO: 103 (depicts the IB1 protein sequence from rat encoded by the exon-intron boundary of the rIB1 gene—splice donor), SEQ ID NO: 104 (depicts the IB1 protein sequence from Homo sapiens), or SEQ ID NO: 105 (depicts the IB1 cDNA sequence from Homo sapiens), more preferably from an amino acid sequence as defined or encoded by any of sequences according to SEQ ID NO: 104 (depicts the IB1 protein sequence from Homo sapiens), or SEQ ID NO: 105 (depicts the IB1 cDNA sequence from Homo sapiens), or from any fragments or variants thereof. In other words, the JNK inhibitor (poly-)peptide comprises a fragment, variant, or variant of such fragment of a human or rat IB1 sequence. Human or rat IB sequences are defined or encoded, respectively, by the sequences according to SEQ ID NO: 102, SEQ ID NO: 103, SEQ ID NO: 104 or SEQ ID NO: 105.

Preferably, such a JNK inhibitor (poly-)peptide as used herein comprises a total length of less than 150 amino acid residues, preferably a range of 5 to 150 amino acid residues, more preferably 10 to 100 amino acid residues, even more preferably 10 to 75 amino acid residues and most preferably a range of 10 to 50 amino acid residues, e.g. 10 to 30, 10 to 20, or 10 to 15 amino acid residues.

More preferably, such a JNK inhibitor (poly-)peptide and the above ranges may be selected from any of the above mentioned sequences, even more preferably from an amino acid sequence as defined according to SEQ ID NO: 104 or as encoded by SEQ ID NO: 105, even more preferably in the region between nucleotides 420 and 980 of SEQ ID NO: 105 or amino acids 105 and 291 of SEQ ID NO: 104, and most preferably in the region between nucleotides 561 and 647 of SEQ ID NO: 105 or amino acids 152 and 180 of SEQ ID NO: 104.

According to a particular embodiment, a JNK inhibitor (poly-)peptide as used herein typically binds JNK and/or inhibits the activation of at least one JNK activated transcription factor, e.g. c-Jun or ATF2 (see e.g. SEQ ID NOs: 15 and 16, respectively) or Elk1.

Likewise, the JNK inhibitor (poly-)peptide as used herein preferably comprises or consists of at least one amino acid sequence according to any one of SEQ ID NOs: 1 to 4, 13 to 20 and 33 to 100, or a fragment, derivative or variant thereof. More preferably, the JNK inhibitor (poly-)peptide as used herein may contain 1, 2, 3, 4 or even more copies of an amino acid sequence according to SEQ ID NOs: 1 to 4, 13 to 20 and 33 to 100, or a variant, fragment or derivative thereof. If present in more than one copy, these amino acid sequences according to SEQ ID NOs: 1 to 4, 13 to 20 and 33 to 100, or variants, fragments, or derivatives thereof as used herein may be directly linked with each other without any linker sequence or via a linker sequence comprising 1 to 10, preferably 1 to 5 amino acids. Amino acids forming the linker sequence are preferably selected from glycine or proline as amino acid residues. More preferably, these amino acid sequences according to SEQ ID NOs: 1 to 4, 13 to 20 and 33 to 100, or fragments, variants or derivatives thereof, as used herein, may be separated by each other by a hinge of two, three or more proline residues.

The JNK inhibitor (poly-)peptides as used herein may be composed of L-amino acids, D-amino acids, or a combination of both. Preferably, the JNK inhibitor (poly-)peptides as used herein comprise at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the JNK inhibitor sequences as used herein in a blockwise, a non-blockwise or in an alternate manner.

According to one preferred embodiment the JNK inhibitor (poly-)peptides as used herein may be exclusively composed of L-amino acids. The JNK inhibitor (poly-)peptides as used herein may then comprise or consist of at least one “native JNK inhibitor sequence” according to SEQ ID NO: 1 or 3. In this context, the term “native” or “native JNK inhibitor sequence(s)” is referred to non-altered JNK inhibitor (poly-)peptide sequences according to any of SEQ ID NOs: 1 or 3, as used herein, entirely composed of L-amino acids.

Accordingly, the JNK inhibitor (poly-)peptide as used herein may comprise or consist of at least one (native) amino acid sequence NH₂-Xₙ^{b}-RPTTLXLXXXXXXXQD-Xₙ^{a}-Xₙ^{b}-COOH (L-IB generic (s)) [SEQ ID NO: 3] and/or the JNK binding domain (JBDs) of IB1 XRPTTLXLXXXXXXXQDS/TX (L-IB (generic)) [SEQ ID NO: 19]. In this context, each X typically represents an amino acid residue, preferably selected from any (native) amino acid residue. Xₙ^{a} typically represents one amino acid residue, preferably selected from any amino acid residue except serine or threonine, wherein n (the number of repetitions of X) is 0 or 1. Furthermore, each Xₙ^{b} may be selected individually from any amino acid residue, wherein n (the number of repetitions of X) is 0-5, 5-10, 10-15, 15-20, 20-30 or more, provided that if n (the number of repetitions of X) is 0 for Xₙ^{a}, the directly adjacent Xₙ^{b} does preferably not comprise a serine or threonine at its N-terminus, in order to avoid a serine or threonine at this position. Preferably, Xₙ^{b} represents a contiguous stretch of peptide residues derived from SEQ ID NO: 1 or 3. Xₙ^{a} and Xₙ^{b} may represent either D or L amino acids. Additionally, the JNK inhibitor (poly-)peptide as used herein may comprise or consist of at least one (native) amino acid sequence selected from the group comprising the JNK binding domain of IB1 DTYRPKRPTTLNLFPQVPRSQDT (L-IB1) [SEQ ID NO: 17]. More preferably, the JNK inhibitor (poly-)peptide as used herein further may comprise or consist of at least one (native) amino acid sequence NH₂—RPKRPTTLNLFPQVPRSQD-COOH (L-IB1(s)) [SEQ ID NO: 1]. Furthermore, the JNK inhibitor (poly-)peptide as used herein may comprise or consist of at least one (native) amino acid sequence selected from the group comprising the JNK binding domain of IB1 L-IB1(s1) (NH₂-TLNLFPQVPRSQD-COOH, SEQ ID NO: 33); L-IB1(s2) (NH₂-TTLNLFPQVPRSQ-COOH, SEQ ID NO: 34); L-IB1(s3) (NH₂-PTTLNLFPQVPRS-COOH, SEQ ID NO: 35); L-IB1(s4) (NH₂-RPTTLNLFPQVPR-COOH, SEQ ID NO: 36); L-IB1(s5) (NH₂-KRPTTLNLFPQVP-COOH, SEQ ID NO: 37); L-IB1(s6) (NH₂-PKRPTTLNLFPQV-COOH, SEQ ID NO: 38); L-IB1(s7) (NH₂-RPKRPTTLNLFPQ-COOH, SEQ ID NO: 39); L-IB1(s8) (NH₂-LNLFPQVPRSQD-COOH, SEQ ID NO: 40); L-IB1(s9) (NH₂-TLNLFPQVPRSQ-COOH, SEQ ID NO: 41); L-IB1(s10) (NH₂-TTLNLFPQVPRS-COOH, SEQ ID NO: 42); L-IB1(s11) (NH₂-PTTLNLFPQVPR-COOH, SEQ ID NO: 43); L-IB1(s12) (NH₂-RPTTLNLFPQVP-COOH, SEQ ID NO: 44); L-IB1(s13) (NH₂-KRPTTLNLFPQV-COOH, SEQ ID NO: 45); L-IB1(s14) (NH₂-PKRPTTLNLFPQ-COOH, SEQ ID NO: 46); L-IB1(s15) (NH₂-RPKRPTTLNLFP-COOH, SEQ ID NO: 47); L-IB1(s16) (NH₂-NLFPQVPRSQD-COOH, SEQ ID NO: 48); L-IB1(s17) (NH₂-LNLFPQVPRSQ-COOH, SEQ ID NO: 49); L-IB1(s18) (NH₂-TLNLFPQVPRS-COOH, SEQ ID NO: 50); L-IB1(s19) (NH₂-TTLNLFPQVPR-COOH, SEQ ID NO: 51); L-IB1(s20) (NH₂-PTTLNLFPQVP-COOH, SEQ ID NO: 52); L-IB1(s21) (NH₂-RPTTLNLFPQV-COOH, SEQ ID NO: 53); L-IB1(s22) (NH₂-KRPTTLNLFPQ-COOH, SEQ ID NO: 54); L-IB1(s23) (NH₂-PKRPTTLNLFP-COOH, SEQ ID NO: 55); L-IB1(s24) (NH₂-RPKRPTTLNLF-COOH, SEQ ID NO: 56); L-IB1(s25) (NH₂-LFPQVPRSQD-COOH, SEQ ID NO: 57); L-IB1(s26) (NH₂-NLFPQVPRSQ-COOH, SEQ ID NO: 58); L-IB1(s27) (NH₂-LNLFPQVPRS-COOH, SEQ ID NO: 59); L-IB1(s28) (NH₂-TLNLFPQVPR-COOH, SEQ ID NO: 60); L-IB1(s29) (NH₂-TTLNLFPQVP-COOH, SEQ ID NO: 61); L-IB1(s30) (NH₂-PTTLNLFPQV-COOH, SEQ ID NO: 62); L-IB1(s31) (NH₂-RPTTLNLFPQ-COOH, SEQ ID NO: 63); L-IB1(s32) (NH₂-KRPTTLNLFP-COOH, SEQ ID NO: 64); L-IB1(s33) (NH₂-PKRPTTLNLF-COOH, SEQ ID NO: 65); and L-IB1(s34) (NH₂-RPKRPTTLNL-COOH, SEQ ID NO: 66).

Additionally, the JNK inhibitor (poly-)peptide as used herein may comprise or consist of at least one (native) amino acid sequence selected from the group comprising the (long) JNK binding domain (JBDs) of IB1 PGTGCGDTYRPKRPTTLNLFPQVPRSQDT (IB1-long) [SEQ ID NO: 13], the (long) JNK binding domain of IB2 IPSPSVEEPHKHRPTTLRLTTLGAQDS (IB2-long) [SEQ ID NO: 14], the JNK binding domain of c-Jun GAYGYSNPKILKQSMTLNLADPVGNLKPH (c-Jun) [SEQ ID NO: 15], the JNK binding domain of ATF2 TNEDHLAVHKHKHEMTLKFGPARNDSVIV (ATF2) [SEQ ID NO: 16] (see e.g. FIGS. 1A-1C). In this context, an alignment revealed a partially conserved 8 amino acid sequence (see e.g. FIG. 1A) and a further comparison of the JBDs of IB1 and IB2 revealed two blocks of seven and three amino acids that are highly conserved between the two sequences.

According to another preferred embodiment the JNK inhibitor (poly-)peptides as used herein may be composed in part or exclusively of D-amino acids as defined above. More preferably, these JNK inhibitor (poly-)peptides composed of D-amino acids are non-native D retro-inverso sequences of the above (native) JNK inhibitor sequences. The term “retro-inverso (poly-)peptides” refers to an isomer of a linear peptide sequence in which the direction of the sequence is reversed and the chirality of each amino acid residue is inverted (see e.g. Jameson et al., Nature, 368, 744-746 (1994); Brady et al., Nature, 368, 692-693 (1994)). The advantage of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence or peptide as used according to the present invention may be converted into an D retro-inverso sequence or peptide by synthesizing a reverse of the sequence or peptide for the corresponding native L-amino acid sequence or peptide.

The D retro-inverso (poly-)peptides as used herein and as defined above have a variety of useful properties. For example, D retro-inverso (poly-)peptides as used herein enter cells as efficiently as L-amino acid sequences as used herein, whereas the D retro-inverso sequences as used herein are more stable than the corresponding L-amino acid sequences.

Accordingly, the JNK inhibitor (poly-)peptides as used herein may comprise or consist of at least one D retro-inverso sequence according to the amino acid sequence NH₂-Xₙ^{b}-Xₙ^{a}-DQXXXXXXXLXLTTPR-Xₙ^{b}-COOH (D-IB1 generic (s)) [SEQ ID NO: 4] and/or XS/TDQXXXXXXXLXLTTPRX (D-IB (generic)) [SEQ ID NO: 20]. As used in this context, X, Xₙ^{a} and Xₙ^{b} are as defined above (preferably, representing D amino acids), wherein Xₙ^{b} preferably represents a contiguous stretch of residues derived from SEQ ID NO: 2 or 4. If n is 0 for Xₙ^{a}, the directly adjacent Xₙ^{b} does preferably not comprise a serine or threonine at its C-terminus. Additionally, the JNK inhibitor (poly-)peptides as used herein may comprise or consist of at least one D retro-inverso sequence according to the amino acid sequence comprising the JNK binding domain (JBDs) of IB1 TDQSRPVQPFLNLTTPRKPRYTD (D-IB1) [SEQ ID NO: 18]. More preferably, the JNK inhibitor (poly-)peptides as used herein may comprise or consist of at least one D retro-inverso sequence according to the amino acid sequence NH₂-DQSRPVQPFLNLTTPRKPR-COOH (D-IB1(s)) [SEQ ID NO: 2]. Furthermore, the JNK inhibitor (poly-)peptides as used herein may comprise or consist of at least one D retro-inverso sequence according to the amino acid sequence comprising the JNK binding domain (JBDs) of IB1 D-IB1(s1) (NH₂-QPFLNLTTPRKPR-COOH, SEQ ID NO: 67); D-IB1(s2) (NH₂-VQPFLNLTTPRKP-COOH, SEQ ID NO: 68); D-IB1(s3) (NH₂-PVQPFLNLTTPRK-COOH, SEQ ID NO: 69); D-IB1(s4) (NH₂-RPVQPFLNLTTPR-COOH, SEQ ID NO: 70); D-IB1(s5) (NH₂-SRPVQPFLNLTTP-COOH, SEQ ID NO: 71); D-IB1(s6) (NH₂-QSRPVQPFLNLTT-COOH, SEQ ID NO: 72); D-IB1(s7) (NH₂-DQSRPVQPFLNLT-COOH, SEQ ID NO: 73); D-IB1(s8) (NH₂-PFLNLTTPRKPR-COOH, SEQ ID NO: 74); D-IB1(s9) (NH₂-QPFLNLTTPRKP-COOH, SEQ ID NO: 75); D-IB1(s10) (NH₂-VQPFLNLTTPRK-COOH, SEQ ID NO: 76); D-IB1(s11) (NH₂-PVQPFLNLTTPR-COOH, SEQ ID NO: 77); D-IB1(s12) (NH₂-RPVQPFLNLTTP-COOH, SEQ ID NO: 78); D-IB1(s13) (NH₂-SRPVQPFLNLTT-COOH, SEQ ID NO: 79); D-IB1(s14) (NH₂-QSRPVQPFLNLT-COOH, SEQ ID NO: 80); D-IB1(s15) (NH₂-DQSRPVQPFLNL-COOH, SEQ ID NO: 81); D-IB1(s16) (NH₂-FLNLTTPRKPR-COOH, SEQ ID NO: 82); D-IB1(s17) (NH₂-PFLNLTTPRKP-COOH, SEQ ID NO: 83); D-IB1(s18) (NH₂-QPFLNLTTPRK-COOH, SEQ ID NO: 84); D-IB1(s19) (NH₂-VQPFLNLTTPR-COOH, SEQ ID NO: 85); D-IB1(s20) (NH₂-PVQPFLNLTTP-COOH, SEQ ID NO: 86); D-IB1(s21) (NH₂-RPVQPFLNLTT-COOH, SEQ ID NO: 87); D-IB1(s22) (NH₂-SRPVQPFLNLT-COOH, SEQ ID NO: 88); D-IB1(s23) (NH₂-QSRPVQPFLNL-COOH, SEQ ID NO: 89); D-IB1(s24) (NH₂-DQSRPVQPFLN-COOH, SEQ ID NO: 90); D-IB1(s25) (NH₂-DQSRPVQPFL-COOH, SEQ ID NO: 91); D-IB1(s26) (NH₂-QSRPVQPFLN-COOH, SEQ ID NO: 92); D-IB1(s27) (NH₂-SRPVQPFLNL-COOH, SEQ ID NO: 93); D-IB1(s28) (NH₂-RPVQPFLNLT-COOH, SEQ ID NO: 94); D-IB1(s29) (NH₂-PVQPFLNLTT-COOH, SEQ ID NO: 95); D-IB1(s30) (NH₂-VQPFLNLTTP-COOH, SEQ ID NO: 96); D-IB1(s31) (NH₂-QPFLNLTTPR-COOH, SEQ ID NO: 97); D-IB1(s32) (NH₂-PFLNLTTPRK-COOH, SEQ ID NO: 98); D-IB1(s33) (NH₂-FLNLTTPRKP-COOH, SEQ ID NO: 99); and D-IB1(s34) (NH₂-LNLTTPRKPR-COOH, SEQ ID NO: 100).

The JNK inhibitor (poly-)peptides as used herein and as disclosed above are presented in Table 1 (SEQ ID NO:s 1-4, 13-20 and 33-100). The table presents the name of the JNK inhibitor (poly-)peptides/sequences as used herein, as well as their sequence identifier number, their length, and amino acid sequence. Furthermore, Table 1 shows sequences as well as their generic formulas, e.g. for SEQ ID NO's: 1, 2, 5, 6, 9 and 11 and SEQ ID NO's: 3, 4, 7, 8, 10 and 12, respectively. Table 1 furthermore discloses the chimeric sequences SEQ ID NOs: 9-12 and 23-32 (see below), L-IB1 sequences SEQ ID NOs: 33 to 66 and D-IB1 sequences SEQ ID NOs: 67 to 100.

TABLE 1

| SEQUENCE/PEPTIDE | SEQ | | |
|---|---|---|---|
| NAME | ID NO | AA | SEQUENCE |
| L-IB1 (s) | 1 | 19 | RPKRPTTLNLFPQVPRSQD |
| | | | (NH₂-RPKRPTTLNLFPQVPRSQD-COOH) |
| D-IB1(s) | 2 | 19 | DQSRPVQPFLNLTTPRKPR |
| | | | (NH₂-DQSRPVQPFLNLTTPRKPR-COOH) |
| L-IB (generic) (s) | 3 | 19 | NH₂-Xₙ^{b}-RPTTLXLXXXXXXXQD-Xₙ^{a}-Xₙ^{b}-COOH |
| D-IB (generic) (s) | 4 | 19 | NH₂-Xₙ^{b}-Xₙ^{a}-DQXXXXXXXLXLTTPR-Xₙ^{b}-COOH |
| L-TAT | 5 | 10 | GRKKRRQRRR |
| | | | (NH₂-GRKKRRQRRR-COOH) |
| D-TAT | 6 | 10 | RRRQRRKKRG |
| | | | (NH₂-RRRQRRKKRG-COOH) |
| L-generic-TAT (s) | 7 | 11 | NH₂-Xₙ^{b}-RKKRRQRRR-Xₙ^{b}-COOH |
| D-generic-TAT (s) | 8 | 11 | NH₂-Xₙ^{b}-RRRQRRKKR-Xₙ^{b}-COOH |
| L-TAT-IB1 (s) | 9 | 31 | GRKKRRQRRRPPRPKRPTTLNLFPQVPRSQD |
| | | | (NH₂-GRKKRRQRRRPPRPKRPTTLNLFPQVPRSQD-COOH) |
| L-TAT-IB (generic) (s) | 10 | 29 | NH₂-Xₙ^{b}-RKKRRQRRR-Xₙ^{b}-RPTTLXLXXXXXXXQD-Xₙ^{a}-Xₙ^{b}-COOH |
| D-TAT-IB1(s) | 11 | 31 | DQSRPVQPFLNLTTPRKPRPPRRRQRRKKRG |
| | | | (NH₂-DQSRPVQPFLNLTTPRKPRPPRRRQRRKKRG-COOH) |
| D-TAT-IB (generic) (s) | 12 | 29 | NH₂-Xₙ^{b}-Xₙ^{a}-DQXXXXXXXLXLTTPR-Xₙ^{b}-RRRQRRKKR-Xₙ^{b}-COOH |
| IB1-long | 13 | 29 | PGTGCGDTYRPKRPTTLNLFPQVPRSQDT |
| | | | (NH₂-PGTGCGDTYRPKRPTTLNLFPQVPRSQDT-COOH) |
| IB2-long | 14 | 27 | IPSPSVEEPHKHRPTTLRLTTLGAQDS |
| | | | (NH₂-IPSPSVEEPHKHRPTTLRLTTLGAQDS-COOH) |
| c-Jun | 15 | 29 | GAYGYSNPKILKQSMTLNLADPVGNLKPH |
| | | | (NH₂-GAYGYSNPKILKQSMTLNLADPVGNLKPH-COOH) |
| ATF2 | 16 | 29 | TNEDHLAVHKHKHEMTLKFGPARNDSVIV |
| | | | (NH₂-TNEDHLAVHKHKHEMTLKFGPARNDSVIV-COOH) |
| L-IB1 | 17 | 23 | DTYRPKRPTTLNLFPQVPRSQDT |
| | | | (NH₂-DTYRPKRPTTLNLFPQVPRSQDT-COOH) |
| D-IB1 | 18 | 23 | TDQSRPVQPFLNLTTPRKPRYTD |
| | | | (NH₂-TDQSRPVQPFLNLTTPRKPRYTD-COOH) |
| L-IB (generic) | 19 | 19 | XRPTTLXLXXXXXXXQDS/TX |
| | | | (NH₂-XRPTTLXLXXXXXXXQDS/TX-COOH) |
| D-IB (generic) | 20 | 19 | XS/TDQXXXXXXXLXLTTPRX |
| | | | (NH₂-XS/TDQXXXXXXXLXLTTPRX-COOH) |
| L-generic-TAT | 21 | 17 | XXXXRKKRRQRRRXXXX |
| | | | (NH₂-XXXXRKKRRQRRRXXXX-COOH) |
| D-generic-TAT | 22 | 17 | XXXXRRRQRRKKRXXXX |
| | | | (NH₂-XXXXRRRQRRKKRXXXX-COOH) |
| L-TAT-IB1 | 23 | 35 | GRKKRRQRRRPPDTYRPKRPTTLNLFPQVPRSQDT |
| | | | (NH₂-GRKKRRQRRRPPDTYRPKRPTTLNLFPQVPRSQDT-COOH) |
| L-TAT-IB (generic) | 24 | 42 | XXXXXXXRKKRRQRRRXXXXXXXXRPTTLXLXXXXXXXQDS/TX |
| | | | (NH₂- |
| | | | XXXXXXXRKKRRQRRRXXXXXXXXRPTTLXLXXXXXXXQDS/TX- |
| | | | COOH) |
| D-TAT-IB1 | 25 | 35 | TDQSRPVQPFLNLTTPRKPRYTDPPRRRQRRKKRG |
| | | | (NH₂-TDQSRPVQPFLNLTTPRKPRYTDPPRRRQRRKKRG-COOH) |
| D-TAT-IB (generic) | 26 | 42 | XT/SDQXXXXXXXLXLTTPRXXXXXXXXRRRQRRKKRXXXXXXX |
| | | | (NH₂- |
| | | | XT/SDQXXXXXXXLXLTTPRXXXXXXXXRRRQRRKKRXXXXXXX- |
| | | | COOH) |
| L-TAT-IB1(s1) | 27 | 30 | RKKRRQRRRPPRPKRPTTLNLFPQVPRSQD |
| | | | (NH₂-RKKRRQRRRPPRPKRPTTLNLFPQVPRSQD-COOH) |
| L-TAT-IB1(s2) | 28 | 30 | GRKKRRQRRRXₙ^{c}RPKRPTTLNLFPQVPRSQD |
| | | | (NH₂-GRKKRRQRRRXₙ^{c}RPKRPTTLNLFPQVPRSQD-COOH) |
| L-TAT-IB1(s3) | 29 | 29 | RKKRRQRRRXₙ^{c}RPKRPTTLNLFPQVPRSQD |
| | | | (NH₂-RKKRRQRRRXₙ^{c}RPKRPTTLNLFPQVPRSQD-COOH) |
| D-TAT-IB1(s1) | 30 | 30 | DQSRPVQPFLNLTTPRKPRPPRRRQRRKKR |
| | | | (NH₂-DQSRPVQPFLNLTTPRKPRPPRRRQRRKKR-COOH) |
| D-TAT-IB1(s2) | 31 | 30 | DQSRPVQPFLNLTTPRKPRXₙ^{c}RRRQRRKKRG |
| | | | (NH₂-DQSRPVQPFLNLTTPRKPRXₙ^{c}RRRQRRKKRG-COOH) |
| D-TAT-IB1(s3) | 32 | 29 | DQSRPVQPFLNLTTPRKPRXₙ^{c}RRRQRRKKR |
| | | | (NH₂-DQSRPVQPFLNLTTPRKPRXₙ^{c}RRRQRRKKR-COOH) |
| L-IB1(s1) | 33 | 13 | TLNLFPQVPRSQD |
| | | | (NH₂-TLNLFPQVPRSQD-COOH) |
| L-IB1(s2) | 34 | 13 | TTLNLFPQVPRSQ |
| | | | (NH₂-TTLNLFPQVPRSQ-COOH) |
| L-IB1(s3) | 35 | 13 | PTTLNLFPQVPRS |
| | | | (NH₂-PTTLNLFPQVPRS-COOH) |
| L-IB1(s4) | 36 | 13 | RPTTLNLFPQVPR |
| | | | (NH₂-RPTTLNLFPQVPR-COOH) |
| L-IB1(s5) | 37 | 13 | KRPTTLNLFPQVP |
| | | | (NH₂-KRPTTLNLFPQVP-COOH) |
| L-IB1(s6) | 38 | 13 | PKRPTTLNLFPQV |
| | | | (NH₂-PKRPTTLNLFPQV-COOH) |
| L-IB1(s7) | 39 | 13 | RPKRPTTLNLFPQ |
| | | | (NH₂-RPKRPTTLNLFPQ-COOH) |
| L-IB1(s8) | 40 | 12 | LNLFPQVPRSQD |
| | | | (NH₂-LNLFPQVPRSQD-COOH) |
| L-IB1(s9) | 41 | 12 | TLNLFPQVPRSQ |
| | | | (NH₂-TLNLFPQVPRSQ-COOH) |
| L-IB1(s10) | 42 | 12 | TTLNLFPQVPRS |
| | | | (NH₂-TTLNLFPQVPRS-COOH) |
| L-IB1(s11) | 43 | 12 | PTTLNLFPQVPR |
| | | | (NH₂-PTTLNLFPQVPR-COOH) |
| L-IB1(s12) | 44 | 12 | RPTTLNLFPQVP |
| | | | (NH₂-RPTTLNLFPQVP-COOH) |
| L-IB1(s13) | 45 | 12 | KRPTTLNLFPQV |
| | | | (NH₂-KRPTTLNLFPQV-COOH) |
| L-IB1(s14) | 46 | 12 | PKRPTTLNLFPQ |
| | | | (NH₂-PKRPTTLNLFPQ-COOH) |
| L-IB1(s15) | 47 | 12 | RPKRPTTLNLFP |
| | | | (NH₂-RPKRPTTLNLFP-COOH) |
| L-IB1(s16) | 48 | 11 | NLFPQVPRSQD |
| | | | (NH₂-NLFPQVPRSQD-COOH) |
| L-IB1(s17) | 49 | 11 | LNLFPQVPRSQ |
| | | | (NH₂-LNLFPQVPRSQ-COOH) |
| L-IB1(s18) | 50 | 11 | TLNLFPQVPRS |
| | | | (NH₂-TLNLFPQVPRS-COOH) |
| L-IB1(s19) | 51 | 11 | TTLNLFPQVPR |
| | | | (NH₂-TTLNLFPQVPR-COOH) |
| L-IB1(s20) | 52 | 11 | PTTLNLFPQVP |
| | | | (NH₂-PTTLNLFPQVP-COOH) |
| L-IB1(s21) | 53 | 11 | RPTTLNLFPQV |
| | | | (NH₂-RPTTLNLFPQV-COOH) |
| L-IB1(s22) | 54 | 11 | KRPTTLNLFPQ |
| | | | (NH₂-KRPTTLNLFPQ-COOH) |
| L-IB1(s23) | 55 | 11 | PKRPTTLNLFP |
| | | | (NH₂-PKRPTTLNLFP-COOH) |
| L-IB1(s24) | 56 | 11 | RPKRPTTLNLF |
| | | | (NH₂-RPKRPTTLNLF-COOH) |
| L-IB1(s25) | 57 | 10 | LFPQVPRSQD |
| | | | (NH₂-LFPQVPRSQD-COOH) |
| L-IB1(s26) | 58 | 10 | NLFPQVPRSQ |
| | | | (NH₂-NLFPQVPRSQ-COOH) |
| L-IB1(s27) | 59 | 10 | LNLFPQVPRS |
| | | | (NH₂-LNLFPQVPRS-COOH) |
| L-IB1(s28) | 60 | 10 | TLNLFPQVPR |
| | | | (NH₂-TLNLFPQVPR-COOH) |
| L-IB1(s29) | 61 | 10 | TTLNLFPQVP |
| | | | (NH₂-TTLNLFPQVP-COOH) |
| L-IB1(s30) | 62 | 10 | PTTLNLFPQV |
| | | | (NH₂-PTTLNLFPQV-COOH) |
| L-IB1(s31) | 63 | 10 | RPTTLNLFPQ |
| | | | (NH₂-RPTTLNLFPQ-COOH) |
| L-IB1(s32) | 64 | 10 | KRPTTLNLFP |
| | | | (NH₂-KRPTTLNLFP-COOH) |
| L-IB1(s33) | 65 | 10 | PKRPTTLNLF |
| | | | (NH₂-PKRPTTLNLF-COOH) |
| L-IB1(s34) | 66 | 10 | RPKRPTTLNL |
| | | | (NH₂-RPKRPTTLNL-COOH) |
| D-IB1(s1) | 67 | 13 | QPFLNLTTPRKPR |
| | | | (NH₂-QPFLNLTTPRKPR-COOH) |
| D-IB1(s2) | 68 | 13 | VQPFLNLTTPRKP |
| | | | (NH₂-VQPFLNLTTPRKP-COOH) |
| D-IB1(s3) | 69 | 13 | PVQPFLNLTTPRK |
| | | | (NH₂-PVQPFLNLTTPRK-COOH) |
| D-IB1(s4) | 70 | 13 | RPVQPFLNLTTPR |
| | | | (NH₂-RPVQPFLNLTTPR-COOH) |
| D-IB1(s5) | 71 | 13 | SRPVQPFLNLTTP |
| | | | (NH₂-SRPVQPFLNLTTP-COOH) |
| D-IB1(s6) | 72 | 13 | QSRPVQPFLNLTT |
| | | | (NH₂-QSRPVQPFLNLTT-COOH) |
| D-IB1(s7) | 73 | 13 | DQSRPVQPFLNLT |
| | | | (NH₂-DQSRPVQPFLNLT-COOH) |
| D-IB1(s8) | 74 | 12 | PFLNLTTPRKPR |
| | | | (NH₂-PFLNLTTPRKPR-COOH) |
| D-IB1(s9) | 75 | 12 | QPFLNLTTPRKP |
| | | | (NH₂-QPFLNLTTPRKP-COOH) |
| D-IB1(s10) | 76 | 12 | VQPFLNLTTPRK |
| | | | (NH₂-VQPFLNLTTPRK-COOH) |
| D-IB1(s11) | 77 | 12 | PVQPFLNLTTPR |
| | | | (NH₂-PVQPFLNLTTPR-COOH) |
| D-IB1(s12) | 78 | 12 | RPVQPFLNLTTP |
| | | | (NH₂-RPVQPFLNLTTP-COOH) |
| D-IB1(s13) | 79 | 12 | SRPVQPFLNLTT |
| | | | (NH₂-SRPVQPFLNLTT-COOH) |
| D-IB1(s14) | 80 | 12 | QSRPVQPFLNLT |
| | | | (NH₂-QSRPVQPFLNLT-COOH) |
| D-IB1(s15) | 81 | 12 | DQSRPVQPFLNL |
| | | | (NH₂-DQSRPVQPFLNL-COOH) |
| D-IB1(s16) | 82 | 11 | FLNLTTPRKPR |
| | | | (NH₂-FLNLTTPRKPR-COOH) |
| D-IB1(s17) | 83 | 11 | PFLNLTTPRKP |
| | | | (NH₂-PFLNLTTPRKP-COOH) |
| D-IB1(s18) | 84 | 11 | QPFLNLTTPRK |
| | | | (NH₂-QPFLNLTTPRK-COOH) |
| D-IB1(s19) | 85 | 11 | VQPFLNLTTPR |
| | | | (NH₂-VQPFLNLTTPR-COOH) |
| D-IB1(s20) | 86 | 11 | PVQPFLNLTTP |
| | | | (NH₂-PVQPFLNLTTP-COOH) |
| D-IB1(s21) | 87 | 11 | RPVQPFLNLTT |
| | | | (NH₂-RPVQPFLNLTT-COOH) |
| D-IB1(s22) | 88 | 11 | SRPVQPFLNLT |
| | | | (NH₂-SRPVQPFLNLT-COOH) |
| D-IB1(s23) | 89 | 11 | QSRPVQPFLNL |
| | | | (NH₂-QSRPVQPFLNL-COOH) |
| D-IB1(s24) | 90 | 11 | DQSRPVQPFLN |
| | | | (NH₂-DQSRPVQPFLN-COOH) |
| D-IB1(s25) | 91 | 10 | DQSRPVQPFL |
| | | | (NH₂-DQSRPVQPFL-COOH) |
| D-IB1(s26) | 92 | 10 | QSRPVQPFLN |
| | | | (NH₂-QSRPVQPFLN-COOH) |
| D-IB1(s27) | 93 | 10 | SRPVQPFLNL |
| | | | (NH₂-SRPVQPFLNL-COOH) |
| D-IB1(s28) | 94 | 10 | RPVQPFLNLT |
| | | | (NH₂-RPVQPFLNLT-COOH) |
| D-IB1(s29) | 95 | 10 | PVQPFLNLTT |
| | | | (NH₂-PVQPFLNLTT-COOH) |
| D-IB1(s30) | 96 | 10 | VQPFLNLTTP |
| | | | (NH₂-VQPFLNLTTP-COOH) |
| D-IB1(s31) | 97 | 10 | QPFLNLTTPR |
| | | | (NH₂-QPFLNLTTPR-COOH) |
| D-IB1(s32) | 98 | 10 | PFLNLTTPRK |
| | | | (NH₂-PFLNLTTPRK-COOH) |
| D-IB1(s33) | 99 | 10 | FLNLTTPRKP |
| | | | (NH₂-FLNLTTPRKP-COOH) |
| D-IB1(s34) | 100 | 10 | LNLTTPRKPR |
| | | | (NH₂-LNLTTPRKPR-COOH) |

It will be understood by a person skilled in the art that a given sequence herein which is composed exclusively of D-amino acids is identified by “D-name”. For example, SEQ ID NO:100 has the sequence/peptide name “D-IB1 (s34)”. The given amino acid sequence is LNLTTPRKPR. However, all amino acids are here D-amino acids.

It will be also understood by a person skilled in the art that the terms “entirely composed of L-amino acids”; “exclusively composed of D-amino acids” “entirely composed of D-amino acids” and/or “exclusively composed of D-amino acids” and the like refer to sequences which need not (but may) exclude the presence of glycine residues. Glycine is the only amino acid which is non-chiral. Therefore, the terms “entirely composed of L-amino acids”; “exclusively composed of D-amino acids” “entirely composed of D-amino acids” and/or “exclusively composed of D-amino acids” are intended to make clear that L-amino acids or D-amino acids, respectively, are used where possible. Nevertheless, if presence of a glycine is necessary or favored at a given position in the amino acid sequence, then it may remain there. A good example is L-TAT (SEQ ID NO:5). As used herein said sequence is considered to be exclusively composed of L-amino acids “although” said sequence comprises a non chiral glycine residue. Likewise, D-TAT (SEQ ID NO:6), as used herein, may be considered to be exclusively composed of D-amino acids “although” said sequence comprises a non chiral glycine residue.

According to another preferred embodiment, the JNK inhibitor (poly-)peptide as used herein comprises or consists of at least one variant, fragment and/or derivative of the above defined native or non-native amino acid sequences according to SEQ ID NOs: 1-4, 13-20 and 33-100. Preferably, these variants, fragments and/or derivatives retain biological activity of the above disclosed native or non-native JNK inhibitor (poly-)peptides as used herein, particularly of native or non-native amino acid sequences according to SEQ ID NOs: 1-4, 13-20 and 33-100, i.e. binding JNK and/or inhibiting the activation of at least one JNK activated transcription factor, e.g. c-Jun, ATF2 or Elk1. Functionality may be tested by various tests, e.g. binding tests of the peptide to its target molecule or by biophysical methods, e.g. spectroscopy, computer modeling, structural analysis, etc. Particularly, an JNK inhibitor (poly-)peptide or variants, fragments and/or derivatives thereof as defined above may be analyzed by hydrophilicity analysis (see e.g. Hopp and Woods, 1981. Proc Natl Acad Sci USA 78: 3824-3828) that can be utilized to identify the hydrophobic and hydrophilic regions of the peptides, thus aiding in the design of substrates for experimental manipulation, such as in binding experiments, or for antibody synthesis. Secondary structural analysis may also be performed to identify regions of an JNK inhibitor (poly-)peptide or of variants, fragments and/or derivatives thereof as used herein that assume specific structural motifs (see e.g. Chou and Fasman, 1974, Biochem 13: 222-223). Manipulation, translation, secondary structure prediction, hydrophilicity and hydrophobicity profiles, open reading frame prediction and plotting, and determination of sequence homologies can be accomplished using computer software programs available in the art. Other methods of structural analysis include, e.g. X-ray crystallography (see e.g. Engstrom, 1974. Biochem Exp Biol 11: 7-13), mass spectroscopy and gas chromatography (see e.g. METHODS IN PROTEIN SCIENCE, 1997, J. Wiley and Sons, New York, N.Y.) and computer modeling (see e.g. Fletterick and Zoller, eds., 1986. Computer Graphics and Molecular Modeling, In: CURRENT COMMUNICATIONS IN MOLECULAR BIOLOGY, Cold Spring Harbor Laboratory Press, Cold Spring Harbor, N.Y.) may also be employed.

Accordingly, the JNK inhibitor (poly-)peptide as used herein may comprise or consist of at least one variant of (native or non-native) amino acid sequences according to SEQ ID NOs: 1-4, 13-20 and 33-100. In the context of the present invention, a “variant of a (native or non-native) amino acid sequence according to SEQ ID NOs: 1-4, 13-20 and 33-100” is preferably a sequence derived from any of the sequences according to SEQ ID NOs: 1-4, 13-20 and 33-100, wherein the variant comprises amino acid alterations of the amino acid sequences according to SEQ ID NOs: 1-4, 13-20 and 33-100. Such alterations typically comprise 1 to 20, preferably 1 to 10 and more preferably 1 to 5 substitutions, additions and/or deletions of amino acids according to SEQ ID NOs: 1-4, 13-20 and 33-100, wherein the variant exhibits a sequence identity with any of the sequences according to SEQ ID NOs: 1-4, 13-20 and 33-100 of at least about 30%, 50%, 70%, 80%, 90%, 95%, 98% or even at least about 99%.

If variants of (native or non-native) amino acid sequences according to SEQ ID NOs: 1-4, 13-20 and 33-100 as defined above and used herein are obtained by substitution of specific amino acids, such substitutions preferably comprise conservative amino acid substitutions. Conservative amino acid substitutions may include synonymous amino acid residues within a group which have sufficiently similar physicochemical properties, so that a substitution between members of the group will preserve the biological activity of the molecule (see e.g. Grantham, R. (1974), Science 185, 862-864). It is evident to the skilled person that amino acids may also be inserted and/or deleted in the above-defined sequences without altering their function, particularly if the insertions and/or deletions only involve a few amino acids, e.g. less than twenty, and preferably less than ten, and do not remove or displace amino acids which are critical to functional activity. Moreover, substitutions shall be avoided in variants as used herein, which lead to additional threonines at amino acid positions which are accessible for a phosphorylase, preferably a kinase, in order to avoid inactivation of the JNK-inhibitor (poly-)peptide as used herein or of the chimeric peptide as used herein in vivo or in vitro.

Preferably, synonymous amino acid residues, which are classified into the same groups and are typically exchangeable by conservative amino acid substitutions, are defined in Table 2.

TABLE 2
Preferred Groups of Synonymous
Amino Acid Residues

| Amino | Synonymous |
|---|---|
| Acid | Residue |
| Ser | Ser, Thr, Gly, Asn |
| Arg | Arg, Gln, Lys, Glu, His |
| Leu | Ile, Phe, Tyr, Met, |
| | Val, Leu |
| Pro | Gly, Ala, (Thr), Pro |
| Thr | Pro, Ser, Ala, Gly, His, |
| | Gln, Thr |
| Ala | Gly, Thr, Pro, Ala |
| Val | Met, Tyr, Phe, Ile, |
| | Leu, Val |
| Gly | Ala, (Thr), Pro, Ser, Gly |
| Ile | Met, Tyr, Phe, Val, |
| | Leu, Ile |
| Phe | Trp, Met, Tyr, Ile, Val, |
| | Leu, Phe |
| Tyr | Trp, Met, Phe, Ile, Val, |
| | Leu, Tyr |
| Cys | Ser, Thr, Cys |
| His | Glu, Lys, Gln, Thr, |
| | Arg, His |
| Gln | Glu, Lys, Asn, His, |
| | (Thr), Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, |
| | His, Arg, Glu |
| Met | Phe, Ile, Val, Leu, Met |
| Trp | Trp |

A specific form of a variant of SEQ ID NOs: 1-4, 13-20 and 33-100 as used herein is a fragment of the (native or non-native) amino acid sequences according to SEQ ID NOs: 1, 1-4, 13-20 and 33-100” as used herein, which is typically altered by at least one deletion as compared to SEQ ID NOs 1-4, 13-20 and 33-100. Preferably, a fragment comprises at least 4 contiguous amino acids of any of SEQ ID NOs: 1-4, 13-20 and 33-100, a length typically sufficient to allow for specific recognition of an epitope from any of these sequences. Even more preferably, the fragment comprises 4 to 18, 4 to 15, or most preferably 4 to 10 contiguous amino acids of any of SEQ ID NOs: 1-4, 13-20 and 33-100, wherein the lower limit of the range may be 4, or 5, 6, 7, 8, 9, or 10. Deleted amino acids may occur at any position of SEQ ID NOs: 1-4, 13-20 and 33-100, preferably N- or C-terminally.

Furthermore, a fragment of the (native or non-native) amino acid sequences according to SEQ ID NOs: 1-4, 13-20 and 33-100, as described above, may be defined as a sequence sharing a sequence identity with any of the sequences according to SEQ ID NOs: 1-4, 13-20 and 33-100 as used herein of at least about 30%, 50%, 70%, 80%, 90%, 95%, 98%, or even 99%.

The JNK inhibitor (poly-)peptides/sequences as used herein may further comprise or consist of at least one derivative of (native or non-native) amino acid sequences according to SEQ ID NOs: 1-4, 13-20 and 33-100 as defined above. In this context, a “derivative of an (native or non-native) amino acid sequence according to SEQ ID NOs: 1-4, 13-20 and 33-100” is preferably an amino acid sequence derived from any of the sequences according to SEQ ID NOs: 1-4, 13-20 and 33-100, wherein the derivative comprises at least one modified L- or D-amino acid (forming non-natural amino acid(s)), preferably 1 to 20, more preferably 1 to 10, and even more preferably 1 to 5 modified L- or D-amino acids. Derivatives of variants or fragments also fall under the scope of the present invention.

“A modified amino acid” in this respect may be any amino acid which is altered e.g. by different glycosylation in various organisms, by phosphorylation or by labeling specific amino acids. Such a label is then typically selected from the group of labels comprising:
(i) radioactive labels, i.e. radioactive phosphorylation or a radioactive label with sulphur, hydrogen, carbon, nitrogen, etc.;
(ii) colored dyes (e.g. digoxygenin, etc.);
(iii) fluorescent groups (e.g. fluorescein, etc.);
(iv) chemoluminescent groups;
(v) groups for immobilization on a solid phase (e.g. His-tag, biotin, strep-tag, flag-tag, antibodies, antigen, etc.); and
(vi) a combination of labels of two or more of the labels mentioned under (i) to (v).

In the above context, an amino acid sequence having a sequence “sharing a sequence identity” of at least, for example, 95% to a query amino acid sequence of the present invention, is intended to mean that the sequence of the subject amino acid sequence is identical to the query sequence except that the subject amino acid sequence may include up to five amino acid alterations per each 100 amino acids of the query amino acid sequence. In other words, to obtain an amino acid sequence having a sequence of at least 95% identity to a query amino acid sequence, up to 5% (5 of 100) of the amino acid residues in the subject sequence may be inserted or substituted with another amino acid or deleted.

For sequences without exact correspondence, a “% identity” of a first sequence may be determined with respect to a second sequence. In general, these two sequences to be compared are aligned to give a maximum correlation between the sequences. This may include inserting “gaps” in either one or both sequences, to enhance the degree of alignment. A % identity may then be determined over the whole length of each of the sequences being compared (so-called global alignment), that is particularly suitable for sequences of the same or similar length, or over shorter, defined lengths (so-called local alignment), that is more suitable for sequences of unequal length.

Methods for comparing the identity and homology of two or more sequences, particularly as used herein, are well known in the art. Thus for instance, programs available in the Wisconsin Sequence Analysis Package, version 9.1 (Devereux et at, 1984, Nucleic Acids Res. 12, 387-395.), for example the programs BESTFIT and GAP, may be used to determine the % identity between two polynucleotides and the % identity and the % homology between two polypeptide sequences. BESTFIT uses the “local homology” algorithm of (Smith and Waterman (1981), J. Mol. Biol. 147, 195-197.) and finds the best single region of similarity between two sequences. Other programs for determining identity and/or similarity between sequences are also known in the art, for instance the BLAST family of programs (Altschul et al., 1990, J. Mol. Biol. 215, 403-410), accessible through the home page of the NCBI at world wide web site ncbi.nlm.nih.gov) and FASTA (Pearson (1990), Methods Enzymol. 183, 63-98; Pearson and Lipman (1988), Proc. Natl. Acad. Sci. U.S.A 85, 2444-2448.).

JNK-inhibitor (poly-)peptides/sequences as used according to the present invention and as defined above may be obtained or produced by methods well-known in the art, e.g. by chemical synthesis or by genetic engineering methods as discussed below. For example, a peptide corresponding to a portion of an JNK inhibitor sequence as used herein including a desired region of said JNK inhibitor sequence, or that mediates the desired activity in vitro or in vivo, may be synthesized by use of a peptide synthesizer.

JNK inhibitor (poly-)peptide as used herein and as defined above, may be furthermore be modified by a trafficking (poly-)peptide, allowing the JNK inhibitor (poly-)peptide as used herein and as defined above to be transported effectively into the cells. Such modified JNK inhibitor (poly-)peptides are preferably provided and used as chimeric (poly-)peptides.

According to a second aspect the present invention therefore provides the use of a chimeric (poly-)peptide including at least one first domain and at least one second domain, for the preparation of a pharmaceutical composition for treating dry eye syndrome in a subject, wherein the first domain of the chimeric peptide comprises a trafficking sequence, while the second domain of the chimeric (poly-)peptide comprises an JNK inhibitor sequence as defined above, preferably of any of sequences according to SEQ ID NO: 1-4, 13-20 and 33-100 or a derivative or a fragment thereof.

Typically, chimeric (poly-)peptides as used according to the present invention have a length of at least 25 amino acid residues, e.g. 25 to 250 amino acid residues, more preferably 25 to 200 amino acid residues, even more preferably 25 to 150 amino acid residues, 25 to 100 and most preferably amino acid 25 to 50 amino acid residues.

As a first domain the chimeric (poly-)peptide as used herein preferably comprises a trafficking sequence, which is typically selected from any sequence of amino acids that directs a peptide (in which it is present) to a desired cellular destination. Thus, the trafficking sequence, as used herein, typically directs the peptide across the plasma membrane, e.g. from outside the cell, through the plasma membrane, and into the cytoplasm. Alternatively, or in addition, the trafficking sequence may direct the peptide to a desired location within the cell, e.g. the nucleus, the ribosome, the endoplasmic reticulum (ER), a lysosome, or peroxisome, by e.g. combining two components (e.g. a component for cell permeability and a component for nuclear location) or by one single component having e.g. properties of cell membrane transport and targeted e.g. intranuclear transport. The trafficking sequence may additionally comprise another component, which is capable of binding a cytoplasmic component or any other component or compartment of the cell (e.g. endoplasmic reticulum, mitochondria, gloom apparatus, lysosomal vesicles). Accordingly, e.g. the trafficking sequence of the first domain and the JNK inhibitor sequence of the second domain may be localized in the cytoplasm or any other compartment of the cell. This allows to determine localization of the chimeric peptide in the cell upon uptake.

Preferably, the trafficking sequence (being included in the first domain of the chimeric peptide as used herein) has a length of 5 to 150 amino acid sequences, more preferably a length of 5 to 100 and most preferably a length of from 5 to 50, 5 to 30 or even 5 to 15 amino acids.

More preferably, the trafficking sequence (contained in the first domain of the chimeric peptide as used herein) may occur as a continuous amino acid sequence stretch in the first domain. Alternatively, the trafficking sequence in the first domain may be splitted into two or more fragments, wherein all of these fragments resemble the entire trafficking sequence and may be separated from each other by 1 to 10, preferably 1 to 5 amino acids, provided that the trafficking sequence as such retains its carrier properties as disclosed above. These amino acids separating the fragments of the trafficking sequence may e.g. be selected from amino acid sequences differing from the trafficking sequence. Alternatively, the first domain may contain a trafficking sequence composed of more than one component, each component with its own function for the transport of the cargo JNK inhibitor sequence of the second domain to e.g. a specific cell compartment.

The trafficking sequence as defined above may be composed of L-amino acids, D-amino acids, or a combination of both. Preferably, the trafficking sequences (being included in the first domain of the chimeric peptide as used herein) may comprise at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the JNK trafficking sequences in a blockwise, a non-blockwise or in an alternate manner.

According to one alternative embodiment, the trafficking sequence of the chimeric (poly-)peptide as used herein may be exclusively composed of L-amino acids. More preferably, the trafficking sequence of the chimeric peptide as used herein comprises or consists of at least one “native” trafficking sequence as defined above. In this context, the term “native” is referred to non-altered trafficking sequences, entirely composed of L-amino acids.

According to another alternative embodiment the trafficking sequence of the chimeric (poly-)peptide as used herein may be exclusively composed of D-amino acids. More preferably, the trafficking sequence of the chimeric peptide as used herein may comprise a D retro-inverso peptide of the sequences as presented above.

The trafficking sequence of the first domain of the chimeric (poly-)peptide as used herein may be obtained from naturally occurring sources or can be produced by using genetic engineering techniques or chemical synthesis (see e.g. Sambrook, J., Fritsch, E. F., Maniatis, T. (1989) Molecular cloning: A laboratory manual. 2nd edition. Cold Spring Harbor Laboratory Press, Cold Spring Harbor, N.Y.).

Sources for the trafficking sequence of the first domain may be employed including, e.g. native proteins such as e.g. the TAT protein (e.g. as described in U.S. Pat. Nos. 5,804,604 and 5,674,980, each of these references being incorporated herein by reference), VP22 (described in e.g. WO 97/05265; Elliott and O'Hare, Cell 88: 223-233 (1997)), non-viral proteins (Jackson et al, Proc. Natl. Acad. Sci. USA 89: 10691-10695 (1992)), trafficking sequences derived from Antennapedia (e.g. the antennapedia carrier sequence) or from basic peptides, e.g. peptides having a length of 5 to 15 amino acids, preferably 10 to 12 amino acids and comprising at least 80%, more preferably 85% or even 90% basic amino acids, such as e.g. arginine, lysine and/or histidine. Furthermore, variants, fragments and derivatives of one of the native proteins used as trafficking sequences are disclosed herewith. With regard to variants, fragments and derivatives it is referred to the definition given above for JNK inhibitor sequences as used herein. Variants, fragments as well as derivatives are correspondingly defined as set forth above for JNK inhibitor sequences as used herein. Particularly, in the context of the trafficking sequence, a variant or fragment or derivative may be defined as a sequence sharing a sequence identity with one of the native proteins used as trafficking sequences as defined above of at least about 30%, 50%, 70%, 80%, 90%, 95%, 98%, or even 99%.

In a preferred embodiment of the chimeric (poly-)peptide as used herein, the trafficking sequence of the first domain comprises or consists of a sequence derived from the human immunodeficiency virus (HIV)1 TAT protein, particularly some or all of the 86 amino acids that make up the TAT protein.

For a trafficking sequence (being included in the first domain of the chimeric peptide as used herein), partial sequences of the full-length TAT protein may be used forming a functionally effective fragment of a TAT protein, i.e. a TAT peptide that includes the region that mediates entry and uptake into cells. As to whether such a sequence is a functionally effective fragment of the TAT protein can be determined using known techniques (see e.g. Franked et al., Proc. Natl. Acad. Sci, USA 86: 7397-7401 (1989)). Thus, the trafficking sequence in the first domain of the chimeric peptide as used herein may be derived from a functionally effective fragment or portion of a TAT protein sequence that comprises less than 86 amino acids, and which exhibits uptake into cells, and optionally the uptake into the cell nucleus. More preferably, partial sequences (fragments) of TAT to be used as carrier to mediate permeation of the chimeric peptide across the cell membrane, are intended to comprise the basic region (amino acids 48 to 57 or 49 to 57) of full-length TAT.

According to a more preferred embodiment, the trafficking sequence (being included in the first domain of the chimeric peptide as used herein) may comprise or consist of an amino acid sequence containing TAT residues 48-57 or 49 to 57, and most preferably a generic TAT sequence NH₂-Xₙ^{b}-RKKRRQRRR-Xₙ^{b}-COOH (L-generic-TAT (s)) [SEQ ID NO: 7] and/or XXXXRKKRRQ RRRXXXX (L-generic-TAT) [SEQ ID NO: 21], wherein X or Xₙ^{b} is as defined above. Furthermore, the number of “Xₙ^{b}” residues in SEQ ID NOs:8 is not limited to the one depicted, and may vary as described above. Alternatively, the trafficking sequence being included in the first domain of the chimeric peptide as used herein may comprise or consist of a peptide containing e.g. the amino acid sequence NH₂-GRKKRRQRRR-COOH (L-TAT) [SEQ ID NO: 5].

According to another more preferred embodiment the trafficking sequence (being included in the first domain of the chimeric peptide as used herein) may comprise a D retro-inverso peptide of the sequences as presented above, i.e. the D retro-inverso sequence of the generic TAT sequence having the sequence NH₂-Xₙ^{b}-RRRQRRKKR-Xₙ^{b}-COOH (D-generic-TAT (s)) [SEQ ID NO: 8] and/or XXXXRRRQRRKKRXXXX (D-generic-TAT) [SEQ ID NO: 22]. Also here, Xₙ^{b} is as defined above (preferably representing D amino acids). Furthermore, the number of “Xₙ^{b}” residues in SEQ ID NOs:8 is not limited to the one depicted, and may vary as described above. Most preferably, the trafficking sequence as used herein may comprise the D retro-inverso sequence NH₂-RRRQRRKKRG-COOH (D-TAT) [SEQ ID NO: 6].

According to another embodiment the trafficking sequence being included in the first domain of the chimeric peptide as used herein may comprise or consist of variants of the trafficking sequences as defined above. A “variant of a trafficking sequence” is preferably a sequence derived from a trafficking sequence as defined above, wherein the variant comprises a modification, for example, addition, (internal) deletion (leading to fragments) and/or substitution of at least one amino acid present in the trafficking sequence as defined above. Such (a) modification(s) typically comprise(s) 1 to 20, preferably 1 to 10 and more preferably 1 to 5 substitutions, additions and/or deletions of amino acids. Furthermore, the variant preferably exhibits a sequence identity with the trafficking sequence as defined above, more preferably with any of SEQ ID NOs: 5 to 8 or 21-22, of at least about 30%, 50%, 70%, 80%, 90%, 95%, 98% or even 99%.

Preferably, such a modification of the trafficking sequence being included in the first domain of the chimeric peptide as used herein leads to a trafficking sequence with increased or decreased stability. Alternatively, variants of the trafficking sequence can be designed to modulate intracellular localization of the chimeric peptide as used herein. When added exogenously, such variants as defined above are typically designed such that the ability of the trafficking sequence to enter cells is retained (i.e. the uptake of the variant of the trafficking sequence into the cell is substantially similar to that of the native protein used a trafficking sequence). For example, alteration of the basic region thought to be important for nuclear localization (see e.g. Dang and Lee, J. Biol. Chem. 264: 18019-18023 (1989); Hauber et al., J. Virol. 63: 1181-1187 (1989); et al., J. Virol. 63: 1-8 (1989)) can result in a cytoplasmic location or partially cytoplasmic location of the trafficking sequence, and therefore, of the JNK inhibitor sequence as component of the chimeric peptide as used herein. Additional to the above, further modifications may be introduced into the variant, e.g. by linking e.g. cholesterol or other lipid moieties to the trafficking sequence to produce a trafficking sequence having increased membrane solubility. Any of the above disclosed variants of the trafficking sequences being included in the first domain of the chimeric peptide as used herein can be produced using techniques typically known to a skilled person (see e.g. Sambrook, J., Fritsch, E. F., Maniatis, T. (1989) Molecular cloning: A laboratory manual. 2nd edition. Cold Spring Harbor Laboratory, Cold Spring Harbor, N.Y.)

As a second domain the chimeric peptide as used herein typically comprises an JNK inhibitor sequence, selected from any of the JNK inhibitor sequences as defined above, including variants, fragments and/or derivatives of these JNK inhibitor sequences.

Both domains, i.e. the first and the second domain(s), of the chimeric peptide as used herein, may be linked such as to form a functional unit. Any method for linking the first and second domain(s) as generally known in the art may be applied.

According to one embodiment, the first and the second domain(s) of the chimeric peptide as used herein are preferably linked by a covalent bond. A covalent bond, as defined herein, may be e.g. a peptide bond, which may be obtained by expressing the chimeric peptide as defined above as a fusion protein. Fusion proteins, as described herein, can be formed and used in ways analogous to or readily adaptable from standard recombinant DNA techniques, as described below. However, both domains may also be linked via side chains or may be linked by a chemical linker moiety.

The first and/or second domains of the chimeric peptide as used herein may occur in one or more copies in said chimeric peptide. If both domains are present in a single copy, the first domain may be linked either to the N-terminal or the C-terminal end of the second domain. If present in multiple copies, the first and second domain(s) may be arranged in any possible order. E.g. the first domain can be present in the chimeric peptide as used herein in a multiple copy number, e.g. in two, three or more copies, which are preferably arranged in consecutive order. Then, the second domain may be present in a single copy occurring at the N- or C-terminus of the sequence comprising the first domain. Alternatively, the second domain may be present in a multiple copy number, e.g. in two, three or more copies, and the first domain may be present in a single copy. According to both alternatives, first and second domain(s) can take any place in a consecutive arrangement. Exemplary arrangements are shown in the following: e.g. first domain-first domain-first domain-second domain; first domain-first domain-second domain-first domain; first domain-second domain-first domain-first domain; or e.g. second domain-first domain-first domain-first domain. It is well understood for a skilled person that these examples are for illustration purposes only and shall not limit the scope of the invention thereto. Thus, the number of copies and the arrangement may be varied as defined initially.

Preferably, the first and second domain(s) may be directly linked with each other without any linker. Alternatively, they may be linked with each other via a linker sequence comprising 1 to 10, preferably 1 to 5 amino acids. Amino acids forming the linker sequence are preferably selected from glycine or proline as amino acid residues. More preferably, the first and second domain(s) may be separated by each other by a hinge of two, three or more proline residues between the first and second domain(s).

The chimeric peptide as defined above and as used herein, comprising at least one first and at least one second domain, may be composed of L-amino acids, D-amino acids, or a combination of both. Therein, each domain (as well as the linkers used) may be composed of L-amino acids, D-amino acids, or a combination of both (e.g. D-TAT and L-IB1(s) or L-TAT and D-IB1(s), etc.). Preferably, the chimeric peptide as used herein may comprise at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the chimeric peptide as used herein in a blockwise, a non-blockwise or in an alternate manner.

According to a specific embodiment the chimeric peptide as used herein comprises or consists of the L-amino acid chimeric peptides according to the generic L-TAT-IB peptide NH₂-Xₙ^{b}-RKKRRQRRR-Xₙ^{b}-RPTTLXLXXXXXXXQD-Xₙ^{a}-Xₙ^{b}-COOH (L-TAT-IB (generic) (s)) [SEQ ID NO: 10], wherein X, Xₙ^{a} and Xₙ^{b} are preferably as defined above. More preferably, the chimeric peptide as used herein comprises or consists of the L-amino acid chimeric peptide NH₂-GRKKRRQRRRPPRPKRPTTLNLFPQVPRSQD-COOH (L-TAT-IB1 (s)) [SEQ ID NO: 9]. Alternatively or additionally, the chimeric peptide as used herein comprises or consists of the L-amino acid chimeric peptide sequence GRKKRRQRRR PPDTYRPKRP TTLNLFPQVP RSQDT (L-TAT-IB1) [SEQ ID NO: 23], or XXXXXXXRKK RRQRRRXXXX XXXXRPTTLX LXXXXXXXQD S/TX (L-TAT-IB generic) [SEQ ID NO: 24], wherein X is preferably also as defined above, or the chimeric peptide as used herein comprises or consists of the L-amino acid chimeric peptide sequence RKKRRQRRRPPRPKRPTTLNLFPQVPRSQD (L-TAT-IB1(s1)) [SEQ ID NO: 27], GRKKRRQRRRXₙ^{c}RPKRPTTLNLFPQVPRSQD (L-TAT-IB1(s2)) [SEQ ID NO: 28], or RKKRRQRRRXₙ^{c}RPKRPTTLNLFPQVPRSQD (L-TAT-IB1(s3)) [SEQ ID NO: 29]. In this context, each X typically represents an amino acid residue as defined above, more preferably Xₙ^{c} represents a contiguous stretch of peptide residues, each X independently selected from each other from glycine or proline, e.g. a monotonic glycine stretch or a monotonic proline stretch, wherein n (the number of repetitions of Xₙ^{c}) is typically 0-5, 5-10, 10-15, 15-20, 20-30 or even more, preferably 0-5 or 5-10. Xₙ^{c} may represent either D or L amino acids.

According to an alternative specific embodiment the chimeric peptide as used herein comprises or consists of D-amino acid chimeric peptides of the above disclosed L-amino acid chimeric peptides. Exemplary D retro-inverso chimeric peptides according to the present invention are e.g. the generic D-TAT-IB peptide NH₂-Xₙ^{b}-Xₙ^{a}-DQXXXXXXXLXLTTPR-Xₙ^{b}-RRRQRRKKR-Xₙ^{b}-COOH (D-TAT-IB (generic) (s)) [SEQ ID NO: 12]. Herein, X, Xₙ^{a} and Xₙ^{b} are preferably as defined above (preferably representing D amino acids). More preferably, the chimeric peptide as used herein comprises or consists of D-amino acid chimeric peptides according to the TAT-IB1 peptide NH₂-DQSRPVQPFLNLTTPRKPRPPRRRQRRKKRG-COOH (D-TAT-IB1(s)) [SEQ ID NO: 11]. Alternatively or additionally, the chimeric peptide as used herein comprises or consists of the D-amino acid chimeric peptide sequence TDQSRPVQPFLNLTTPRKPRYTDPPRRRQRRKKRG (D-TAT-IB1) [SEQ ID NO: 25], or XT/SDQXXXXXXXLXLTTPRXXXXXXXXRRRQRRKKRXXXXXXX (D-TAT-IB generic) [SEQ ID NO: 26], wherein X is preferably also as defined above, or the chimeric peptide as used herein comprises or consists of the D-amino acid chimeric peptide sequence DQSRPVQPFLNLTTPRKPRPPRRRQRRKKR (D-TAT-IB1(s1)) [SEQ ID NO: 30], DQSRPVQPFLNLTTPRKPRXₙ^{c}TRRQRRKKRG (D-TAT-IB1(s2)) [SEQ ID NO: 31], or DQSRPVQPFLNLTTPRKPRXₙ^{c}RRRQRRKKR (D-TAT-IB1(s3)) [SEQ ID NO: 32]. Xₙ^{c} may be as defined above.

The first and second domain(s) of the chimeric peptide as defined above may be linked to each other by chemical or biochemical coupling carried out in any suitable manner known in the art, e.g. by establishing a peptide bond between the first and the second domain(s) e.g. by expressing the first and second domain(s) as a fusion protein, or e.g. by crosslinking the first and second domain(s) of the chimeric peptide as defined above.

Many known methods suitable for chemical crosslinking of the first and second domain(s) of the chimeric peptide as defined above are non-specific, i.e. they do not direct the point of coupling to any particular site on the transport polypeptide or cargo macromolecule. As a result, use of non-specific crosslinking agents may attack functional sites or sterically block active sites, rendering the conjugated proteins biologically inactive. Thus, preferably such crosslinking methods are used, which allow a more specific coupling of the first and second domain(s).

In this context, one way to increasing coupling specificity is a direct chemical coupling to a functional group present only once or a few times in one or both of the first and second domain(s) to be crosslinked. For example, cysteine, which is the only protein amino acid containing a thiol group, occurs in many proteins only a few times. Also, for example, if a polypeptide contains no lysine residues, a crosslinking reagent specific for primary amines will be selective for the amino terminus of that polypeptide. Successful utilization of this approach to increase coupling specificity requires that the polypeptide have the suitably rare and reactive residues in areas of the molecule that may be altered without loss of the molecule's biological activity. Cysteine residues may be replaced when they occur in parts of a polypeptide sequence where their participation in a crosslinking reaction would otherwise likely interfere with biological activity. When a cysteine residue is replaced, it is typically desirable to minimize resulting changes in polypeptide folding. Changes in polypeptide folding are minimized when the replacement is chemically and sterically similar to cysteine. For these reasons, serine is preferred as a replacement for cysteine. As demonstrated in the examples below, a cysteine residue may be introduced into a polypeptide's amino acid sequence for crosslinking purposes. When a cysteine residue is introduced, introduction at or near the amino or carboxy terminus is preferred. Conventional methods are available for such amino acid sequence modifications, wherein the polypeptide of interest is produced by chemical synthesis or via expression of recombinant DNA.

Coupling of the first and second domain(s) of the chimeric peptide as defined above and used herein can also be accomplished via a coupling or conjugating agent. There are several intermolecular crosslinking reagents which can be utilized (see for example, Means and Feeney, CHEMICAL MODIFICATION OF PROTEINS, Holden-Day, 1974, pp. 39-43). Among these reagents are, for example, N-succinimidyl 3-(2-pyridyldithio) propionate (SPDP) or N,N′-(1,3-phenylene) bismaleimide (both of which are highly specific for sulfhydryl groups and form irreversible linkages); N,N′-ethylene-bis-(iodoacetamide) or other such reagent having 6 to 11 carbon methylene bridges (which are relatively specific for sulfhydryl groups); and 1,5-difluoro-2,4-dinitrobenzene (which forms irreversible linkages with amino and tyrosine groups). Other crosslinking reagents useful for this purpose include: p,p′-difluoro-m,m′-dinitrodiphenylsulfone which forms irreversible crosslinkages with amino and phenolic groups); dimethyl adipimidate (which is specific for amino groups); phenol-1,4 disulfonylchloride (which reacts principally with amino groups); hexamethylenediisocyanate or diisothiocyanate, or azophenyl-p-diisocyanate (which reacts principally with amino groups); glutaraldehyde (which reacts with several different side chains) and disdiazobenzidine (which reacts primarily with tyrosine and histidine).

Crosslinking reagents used for crosslinking the first and second domain(s) of the chimeric peptide as defined above may be homobifunctional, i.e. having two functional groups that undergo the same reaction. A preferred homobifunctional crosslinking reagent is bismaleimidohexane (“BMH”). BMH contains two maleimide functional groups, which react specifically with sulfhydryl-containing compounds under mild conditions (pH 6.5-7.7). The two maleimide groups are connected by a hydrocarbon chain. Therefore, BMH is useful for irreversible crosslinking of polypeptides that contain cysteine residues.

Crosslinking reagents used for crosslinking the first and second domain(s) of the chimeric peptide as defined above may also be heterobifunctional. Heterobifunctional crosslinking agents have two different functional groups, for example an amine-reactive group and a thiol-reactive group, that will crosslink two proteins having free amines and thiols, respectively. Examples of heterobifunctional crosslinking agents are succinimidyl 4-(N-maleimidomethyl)cyclohexane-1-carboxylate (“SMCC”), m-maleimidobenzoyl-N-hydroxysuccinimide ester (“MBS”), and succinimide 4-(p-maleimidophenyl)butyrate (“SMPB”), an extended chain analog of MBS. The succinimidyl group of these crosslinkers reacts with a primary amine, and the thiol-reactive maleimide forms a covalent bond with the thiol of a cysteine residue.

Crosslinking reagents suitable for crosslinking the first and second domain(s) of the chimeric peptide as defined above often have low solubility in water. A hydrophilic moiety, such as a sulfonate group, may thus be added to the crosslinking reagent to improve its water solubility. In this respect, Sulfo-MBS and Sulfo-SMCC are examples of crosslinking reagents modified for water solubility, which may be used according to the present invention.

Likewise, many crosslinking reagents yield a conjugate that is essentially non-cleavable under cellular conditions. However, some crosslinking reagents particularly suitable for crosslinking the first and second domain(s) of the chimeric peptide as defined above contain a covalent bond, such as a disulfide, that is cleavable under cellular conditions. For example, Traut's reagent, dithiobis(succinimidylpropionate) (“DSP”), and N-succinimidyl 3-(2-pyridyldithio)propionate (“SPDP”) are well-known cleavable crosslinkers. The use of a cleavable crosslinking reagent permits the cargo moiety to separate from the transport polypeptide after delivery into the target cell. Direct disulfide linkage may also be useful.

Numerous crosslinking reagents, including the ones discussed above, are commercially available. Detailed instructions for their use are readily available from the commercial suppliers. A general reference on protein crosslinking and conjugate preparation is: Wong, CHEMISTRY OF PROTEIN CONJUGATION AND CROSSLINKING, CRC Press (1991).

Chemical crosslinking of the first and second domain(s) of the chimeric peptide as defined above may include the use of spacer arms. Spacer arms provide intramolecular flexibility or adjust intramolecular distances between conjugated moieties and thereby may help preserve biological activity. A spacer arm may be in the form of a polypeptide moiety that includes spacer amino acids, e.g. proline. Alternatively, a spacer arm may be part of the crosslinking reagent, such as in “long-chain SPDP” (Pierce Chem. Co., Rockford, Ill., cat. No. 21651 H).

Furthermore, variants, fragments or derivatives of one of the above disclosed chimeric peptides may be used herein. With regard to fragments and variants it is generally referred to the definition given above for JNK inhibitor sequences.

Particularly, in the context of the present invention, a “variant of a chimeric peptide” is preferably a sequence derived from any of the sequences according to SEQ ID NOs: 9 to 12 and 23 to 32, wherein the chimeric variant comprises amino acid alterations of the chimeric peptides according to SEQ ID NOs: 9 to 12 and 23 to 32 as used herein. Such alterations typically comprise 1 to 20, preferably 1 to 10 and more preferably 1 to 5 substitutions, additions and/or deletions (leading to fragments) of amino acids according to SEQ ID NOs: 9 to 12 and 23 to 32, wherein the altered chimeric peptide as used herein exhibits a sequence identity with any of the sequences according to SEQ ID NOs: 9-12 and 23 to 32 of at least about 30%, 50%, 70%, 80%, or 95%, 98%, or even 99%. Preferably, these variants retain the biological activity of the first and the second domain as contained in the chimeric peptide as used herein, i.e. the trafficking activity of the first domain as disclosed above and the activity of the second domain for binding JNK and/or inhibiting the activation of at least one JNK activated transcription factor.

Accordingly, the chimeric peptide as used herein also comprises fragments of the afore disclosed chimeric peptides, particularly of the chimeric peptide sequences according to any of SEQ ID NOs: 9 to 12 and 23 to 32. Thus, in the context of the present invention, a “fragment of the chimeric peptide” is preferably a sequence derived any of the sequences according to SEQ ID NOs: 9 to 12 and 23 to 32, wherein the fragment comprises at least 4 contiguous amino acids of any of SEQ ID NOs: 9 to 12 and 23 to 32. This fragment preferably comprises a length which is sufficient to allow specific recognition of an epitope from any of these sequences and to transport the sequence into the cells, the nucleus or a further preferred location. Even more preferably, the fragment comprises 4 to 18, 4 to 15, or most preferably 4 to 10 contiguous amino acids of any of SEQ ID NOs: 9 to 12 and 23 to 32. Fragments of the chimeric peptide as used herein further may be defined as a sequence sharing a sequence identity with any of the sequences according to any of SEQ ID NOs: 99 to 12 and 23 to 32 of at least about 30%, 50%, 70%, 80%, or 95%, 98%, or even 99%.

Finally, the chimeric peptide as used herein also comprises derivatives of the afore disclosed chimeric peptides, particularly of the chimeric peptide sequences according to any of SEQ ID NOs: 9 to 12 and 23 to 32.

A particularly preferred use of the present invention is the use of a JNK inhibitor (poly-)peptide consisting of or comprising the amino acid sequence of SEQ ID NO: 11, or consisting of or comprising an amino acid sequence sharing a sequence identity of at least about 30%, 50%, 70%, 80%, 90%, 92% or even 95% with SEQ ID NO: 11, for the treatment of dry eye syndrome. The JNK inhibitor (poly-)peptide consisting of or comprising the amino acid sequence of SEQ ID NO: 11, or consisting of or comprising an amino acid sequence sharing a sequence identity of at least about 30%, 50%, 70%, 80%, 90%, 92% or even 95% with SEQ ID NO: 11 may be administered for example locally to the eye or systemically.

The present invention additionally refers to the use of nucleic acid sequences encoding JNK inhibitor sequences as defined above, chimeric peptides or their fragments, variants or derivatives, all as defined above, for the preparation of a pharmaceutical composition for treating dry eye syndrome in a subject as defined herein. A preferable suitable nucleic acid encoding a JNK inhibitor sequence as used herein is typically chosen from human IB1 nucleic acid (GenBank Accession No. (AF074091), rat IB1 nucleic acid (GenBank Accession No. AF 108959), or human IB2 (GenBank Accession No AF218778) or from any nucleic acid sequence encoding any of the sequences as defined above, i.e. any sequence according to SEQ ID NO: 1-26.

Nucleic acids encoding the JNK inhibitor sequences as used herein or chimeric peptides as used herein may be obtained by any method known in the art (e.g. by PCR amplification using synthetic primers hybridizable to the 3′- and 5′-termini of the sequence and/or by cloning from a cDNA or genomic library using an oligonucleotide sequence specific for the given gene sequence).

Additionally, nucleic acid sequences are disclosed herein as well, which hybridize under stringent conditions with the appropriate strand coding for a (native) JNK inhibitor sequence or chimeric peptide as defined above. Preferably, such nucleic acid sequences comprise at least 6 (contiguous) nucleic acids, which have a length sufficient to allow for specific hybridization. More preferably, such nucleic acid sequences comprise 6 to 38, even more preferably 6 to 30, and most preferably 6 to 20 or 6 to 10 (contiguous) nucleic acids.

“Stringent conditions” are sequence dependent and will be different under different circumstances. Generally, stringent conditions can be selected to be about 5° C. lower than the thermal melting point (TM) for the specific sequence at a defined ionic strength and pH. The TM is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may affect the stringency of hybridization (including, among others, base composition and size of the complementary strands), the presence of organic solvents and the extent of base mismatching, the combination of parameters is more important than the absolute measure of any one.

“High stringency conditions” may comprise the following, e.g. Step 1: Filters containing DNA are pretreated for 8 hours to overnight at 65° C. in buffer composed of 6*SSC, 50 mM Tris-HCl (pH 7.5), 1 mM EDTA, 0.02% PVP, 0.02% Ficoll, 0.02% BSA, and 500 μg/ml denatured salmon sperm DNA. Step 2: Filters are hybridized for 48 hours at 65° C. in the above prehybridization mixture to which is added 100 mg/ml denatured salmon sperm DNA and 5-20*10⁶ cpm of ³²P-labeled probe. Step 3: Filters are washed for 1 hour at 37° C. in a solution containing 2*SSC, 0.01% PVP, 0.01% Ficoll, and 0.01% BSA. This is followed by a wash in 0.1*SSC at 50° C. for 45 minutes. Step 4: Filters are autoradiographed. Other conditions of high stringency that may be used are well known in the art (see e.g. Ausubel et al., (eds.), 1993, Current Protocols in Molecular Biology, John Wiley and Sons, NY; and Kriegler, 1990, Gene Transfer and Expression, a Laboratory Manual, Stockton Press, NY).

“Moderate stringency conditions” can include the following: Step 1: Filters containing DNA are pretreated for 6 hours at 55° C. in a solution containing 6*SSC, 5*Denhardt's solution, 0.5% SDS and 100 mg/ml denatured salmon sperm DNA. Step 2: Filters are hybridized for 18-20 hours at 55° C. in the same solution with 5-20*10⁶ cpm ³²P-labeled probe added. Step 3: Filters are washed at 37° C. for 1 hour in a solution containing 2*SSC, 0.1% SDS, then washed twice for 30 minutes at 60° C. in a solution containing 1*SSC and 0.1% SDS. Step 4: Filters are blotted dry and exposed for autoradiography. Other conditions of moderate stringency that may be used are well-known in the art (see e.g. Ausubel et al., (eds.), 1993, Current Protocols in Molecular Biology, John Wiley and Sons, NY; and Kriegler, 1990, Gene Transfer and Expression, a Laboratory Manual, Stockton Press, NY).

Finally, “low stringency conditions” can include: Step 1: Filters containing DNA are pretreated for 6 hours at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA. Step 2: Filters are hybridized for 18-20 hours at 40° C. in the same solution with the addition of 0.02% PVP, 0.02% Ficoll, 0.2% BSA, 100 μg/ml salmon sperm DNA, 10% (wt/vol) dextran sulfate, and 5-20×106 cpm ³²P-labeled probe. Step 3: Filters are washed for 1.5 hours at 55 C in a solution containing 2×SSC, 25 mM Tris-HCl (pH 7.4), 5 mM EDTA, and 0.1% SDS. The wash solution is replaced with fresh solution and incubated an additional 1.5 hours at 60° C. Step 4: Filters are blotted dry and exposed for autoradiography. If necessary, filters are washed for a third time at 65-68° C. and reexposed to film. Other conditions of low stringency that may be used are well known in the art (e.g. as employed for cross-species hybridizations). See e.g. Ausubel et al., (eds.), 1993, CURRENT PROTOCOLS IN MOLECULAR BIOLOGY, John Wiley and Sons, NY; and Kriegler, 1990, GENE TRANSFER AND EXPRESSION, A LABORATORY MANUAL, Stockton Press, NY.

The nucleic acid sequences as defined above according to the present invention can be used to express peptides, i.e. an JNK inhibitor sequence as used herein or an chimeric peptide as used herein for analysis, characterization or therapeutic use; as markers for tissues in which the corresponding peptides (as used herein) are preferentially expressed (either constitutively or at a particular stage of tissue differentiation or development or in disease states). Other uses for these nucleic acids include, e.g. molecular weight markers in gel electrophoresis-based analysis of nucleic acids.

According to a further embodiment of the present invention, expression vectors may be used for the above purposes for recombinant expression of one or more JNK inhibitor sequences and/or chimeric peptides as defined above. The term “expression vector” is used herein to designate either circular or linear DNA or RNA, which is either double-stranded or single-stranded. It further comprises at least one nucleic acid as defined above to be transferred into a host cell or into a unicellular or multicellular host organism. The expression vector as used herein preferably comprises a nucleic acid as defined above encoding the JNK inhibitor sequence as used herein or a fragment or a variant thereof, or the chimeric peptide as used herein, or a fragment or a variant thereof. Additionally, an expression vector according to the present invention preferably comprises appropriate elements for supporting expression including various regulatory elements, such as enhancers/promoters from viral, bacterial, plant, mammalian, and other eukaryotic sources that drive expression of the inserted polynucleotide in host cells, such as insulators, boundary elements, LCRs (e.g. described by Blackwood and Kadonaga (1998), Science 281, 61-63) or matrix/scaffold attachment regions (e.g. described by Li, Harju and Peterson, (1999), Trends Genet. 15, 403-408). In some embodiments, the regulatory elements are heterologous (i.e. not the native gene promoter). Alternately, the necessary transcriptional and translational signals may also be supplied by the native promoter for the genes and/or their flanking regions.

The term “promoter” as used herein refers to a region of DNA that functions to control the transcription of one or more nucleic acid sequences as defined above, and that is structurally identified by the presence of a binding site for DNA-dependent RNA-polymerase and of other DNA sequences, which interact to regulate promoter function. A functional expression promoting fragment of a promoter is a shortened or truncated promoter sequence retaining the activity as a promoter. Promoter activity may be measured by any assay known in the art (see e.g. Wood, de Wet, Dewji, and DeLuca, (1984), Biochem Biophys. Res. Commun. 124, 592-596; Seliger and McElroy, (1960), Arch. Biochem. Biophys. 88, 136-141) or commercially available from Promega®).

An “enhancer region” to be used in the expression vector as defined herein, typically refers to a region of DNA that functions to increase the transcription of one or more genes. More specifically, the term “enhancer”, as used herein, is a DNA regulatory element that enhances, augments, improves, or ameliorates expression of a gene irrespective of its location and orientation vis-à-vis the gene to be expressed, and may be enhancing, augmenting, improving, or ameliorating expression of more than one promoter.

The promoter/enhancer sequences to be used in the expression vector as defined herein, may utilize plant, animal, insect, or fungus regulatory sequences. For example, promoter/enhancer elements can be used from yeast and other fungi (e.g. the GAL4 promoter, the alcohol dehydrogenase promoter, the phosphoglycerol kinase promoter, the alkaline phosphatase promoter). Alternatively, or in addition, they may include animal transcriptional control regions, e.g. (i) the insulin gene control region active within pancreatic beta-cells (see e.g. Hanahan, et al., 1985. Nature 315: 115-122); (ii) the immunoglobulin gene control region active within lymphoid cells (see e.g. Grosschedl, et al., 1984, Cell 38: 647-658); (iii) the albumin gene control region active within liver (see e.g. Pinckert, et al., 1987. Genes and Dev 1: 268-276; (iv) the myelin basic protein gene control region active within brain oligodendrocyte cells (see e.g. Readhead, et al., 1987, Cell 48: 703-712); and (v) the gonadotropin-releasing hormone gene control region active within the hypothalamus (see e.g. Mason, et al., 1986, Science 234: 1372-1378), and the like.

Additionally, the expression vector as defined herein may comprise an amplification marker. This amplification marker may be selected from the group consisting of, e.g. adenosine deaminase (ADA), dihydrofolate reductase (DHFR), multiple drug resistance gene (MDR), ornithine decarboxylase (ODC) and N-(phosphonacetyl)-L-aspartate resistance (CAD).

Exemplary expression vectors or their derivatives suitable for the present invention particularly include, e.g. human or animal viruses (e.g. vaccinia virus or adenovirus); insect viruses (e.g. baculovirus); yeast vectors; bacteriophage vectors (e.g. lambda phage); plasmid vectors and cosmid vectors.

The present invention additionally may utilize a variety of host-vector systems, which are capable of expressing the peptide coding sequence(s) of nucleic acids as defined above. These include, but are not limited to: (i) mammalian cell systems that are infected with vaccinia virus, adenovirus, and the like; (ii) insect cell systems infected with baculovirus and the like; (iii) yeast containing yeast vectors or (iv) bacteria transformed with bacteriophage, DNA, plasmid DNA, or cosmid DNA. Depending upon the host-vector system utilized, any one of a number of suitable transcription and translation elements may be used.

Preferably, a host cell strain, suitable for such a host-vector system, may be selected that modulates the expression of inserted sequences of interest, or modifies or processes expressed peptides encoded by the sequences in the specific manner desired. In addition, expression from certain promoters may be enhanced in the presence of certain inducers in a selected host strain; thus facilitating control of the expression of a genetically-engineered peptide. Moreover, different host cells possess characteristic and specific mechanisms for the translational and post-translational processing and modification (e.g. glycosylation, phosphorylation, and the like) of expressed peptides. Appropriate cell lines or host systems may thus be chosen to ensure the desired modification and processing of the foreign peptide is achieved. For example, peptide expression within a bacterial system can be used to produce an non-glycosylated core peptide; whereas expression within mammalian cells ensures “native” glycosylation of a heterologous peptide.

The JNK inhibitor sequences, chimeric peptides, nucleic acids, vectors, and/or host cells as defined according to the invention can be formulated in a pharmaceutical composition, which may be applied in the prevention or treatment of any of the diseases as defined herein, particularly in the prevention or treatment of dry eye syndrome as defined herein. Typically, such a pharmaceutical composition used according to the present invention includes as an active component, e.g.: (i) any one or more of the JNK inhibitor sequences and/or chimeric peptides as defined above, and/or variants, fragments or derivatives thereof, particularly JNK inhibitor sequences according to any of sequences of SEQ ID NOs: 1 to 4 and 13 to 20 and 33-100 and/or chimeric peptides according to any of sequences of SEQ ID NOs: 9 to 12 and 23 to 32, and/or JNK inhibitor sequences according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions; and/or (ii) nucleic acids encoding an JNK inhibitor sequence and/or an chimeric peptide as defined above and/or variants or fragments thereof, and/or (iii) cells comprising any one or more of the JNK inhibitor sequences and/or chimeric peptides, and/or variants, fragments or derivatives thereof, as defined above and/or (iv) cells transfected with a vector and/or nucleic acids encoding an JNK inhibitor sequence and/or an chimeric peptide as defined above and/or variants or fragments thereof.

According to a preferred embodiment, such a pharmaceutical composition as used according to the present invention typically comprises a safe and effective amount of a component as defined above, preferably of at least one JNK inhibitor sequence according to any of sequences of SEQ ID NOs: 1 to 4 and 13 to 20 and 33-100 and/or at least one chimeric peptide according to any of sequences of SEQ ID NOs: 9 to 12 and 23 to 32, and/or at least one JNK inhibitor sequence according to any of sequences of SEQ ID NOs: 1 to 4 and 13 to 20 and 33-100 comprising a trafficking sequence according to any of SEQ ID NOs: 5-8 and 21 to 22, or variants or fragments thereof within the above definitions, or at least one nucleic acids encoding same, or at least one vector, or host cell as defined above.

The amount of a JNK-inhibitor sequence and chimeric peptide, respectively, in the pharmaceutical composition to be administered to a subject, may—without being limited thereto—have a very low dose. Thus, the dose may be much lower than for peptide drugs known in the art, such as DTS-108 (Florence Meyer-Losic et al., Clin Cancer Res., 2008, 2145-53). This has several positive aspects, for example a reduction of potential side reactions and a reduction in costs.

Preferably, the dose (per kg bodyweight) is in the range of up to 10 mmol/kg, preferably up to 1 mmol/kg, more preferably up to 100 μmol/kg, even more preferably up to 10 μmol/kg, even more preferably up to 1 μmol/kg, even more preferably up to 100 nmol/kg, most preferably up to 50 nmol/kg.

Thus, the dose range may preferably be from about 1 pmol/kg to about 1 mmol/kg, from about 10 pmol/kg to about 0.1 mmol/kg, from about 10 pmol/kg to about 0.01 mmol/kg, from about 50 pmol/kg to about 1 pmol/kg, from about 100 pmol/kg to about 500 nmol/kg, from about 200 pmol/kg to about 300 nmol/kg, from about 300 pmol/kg to about 100 nmol/kg, from about 500 pmol/kg to about 50 nmol/kg, from about 750 pmol/kg to about 30 nmol/kg, from about 250 pmol/kg to about 5 nmol/kg, from about 1 nmol/kg to about 10 nmol/kg, or a combination of any two of said values.

Exemplary doses of a JNK-inhibitor according to SEQ ID NO: 30 may be for example about 10, 50 or 100 μg/kg.

In this context, prescription of treatment, e.g. decisions on dosage etc. when using the above pharmaceutical composition is typically within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in REMINGTON'S PHARMACEUTICAL SCIENCES, 16th edition, Osol, A. (ed), 1980. Accordingly, a “safe and effective amount” as defined above for components of the pharmaceutical compositions as used according to the present invention means an amount of each or all of these components, that is sufficient to significantly induce a positive modification of dry eye syndrome. At the same time, however, a “safe and effective amount” is small enough to avoid serious side-effects, that is to say to permit a sensible relationship between advantage and risk. The determination of these limits typically lies within the scope of sensible medical judgment. A “safe and effective amount” of such a component will vary in connection with the particular condition to be treated and also with the age and physical condition of the patient to be treated, the severity of the condition, the duration of the treatment, the nature of the accompanying therapy, of the particular pharmaceutically acceptable carrier used, and similar factors, within the knowledge and experience of the accompanying doctor. The pharmaceutical compositions according to the invention can be used according to the invention for human and also for veterinary medical purposes.

The pharmaceutical composition as used according to the present invention may furthermore comprise, in addition to one of these substances, a (compatible) pharmaceutically acceptable carrier, excipient, buffer, stabilizer or other materials well known to those skilled in the art.

In this context, the expression “(compatible) pharmaceutically acceptable carrier” preferably includes the liquid or non-liquid basis of the composition. The term “compatible” means that the constituents of the pharmaceutical composition as used herein are capable of being mixed with the pharmaceutically active component as defined above and with one another component in such a manner that no interaction occurs which would substantially reduce the pharmaceutical effectiveness of the composition under usual use conditions. Pharmaceutically acceptable carriers must, of course, have sufficiently high purity and sufficiently low toxicity to make them suitable for administration to a person to be treated.

If the pharmaceutical composition as used herein is provided in liquid form, the pharmaceutically acceptable carrier will typically comprise one or more (compatible) pharmaceutically acceptable liquid carriers. The composition may comprise as (compatible) pharmaceutically acceptable liquid carriers e.g. pyrogen-free water; isotonic saline or buffered (aqueous) solutions, e.g. phosphate, citrate etc. buffered solutions, vegetable oils, such as, for example, groundnut oil, cottonseed oil, sesame oil, olive oil, corn oil and oil from theobroma; polyols, such as, for example, polypropylene glycol, glycerol, sorbitol, mannitol and polyethylene glycol; alginic acid, etc. Particularly for injection of the pharmaceutical composition as used herein, a buffer, preferably an aqueous buffer, may be used.

If the pharmaceutical composition as used herein is provided in solid form, the pharmaceutically acceptable carrier will typically comprise one or more (compatible) pharmaceutically acceptable solid carriers. The composition may comprise as (compatible) pharmaceutically acceptable solid carriers e.g. one or more compatible solid or liquid fillers or diluents or encapsulating compounds may be used as well, which are suitable for administration to a person. Some examples of such (compatible) pharmaceutically acceptable solid carriers are e.g. sugars, such as, for example, lactose, glucose and sucrose; starches, such as, for example, corn starch or potato starch; cellulose and its derivatives, such as, for example, sodium carboxymethylcellulose, ethylcellulose, cellulose acetate; powdered tragacanth; malt; gelatin; tallow; solid glidants, such as, for example, stearic acid, magnesium stearate; calcium sulphate, etc.

The precise nature of the (compatible) pharmaceutically acceptable carrier or other material may depend on the route of administration. The choice of a (compatible) pharmaceutically acceptable carrier may thus be determined in principle by the manner in which the pharmaceutical composition as used according to the invention is administered. The pharmaceutical composition as used according to the invention can be administered, for example, systemically. Routes for administration include, for example, parenteral routes (e.g. via injection), such as intravenous, intramuscular, subcutaneous, intradermal, or transdermal routes, etc., enteral routes, such as oral, or rectal routes, etc., topical routes, such as nasal, or intranasal routes, etc., or other routes, such as epidermal routes or patch delivery. Particularly preferred is also the local administration at/in the eye, e.g. intravitreous administration, subconjuntival administration and/or instillation. Instillation is the most preferred route of administration for the treatment of dry eye as discussed herein, in particular if a JNK inhibitor peptide like SEQ ID NO:11 is used.

In a further aspect the JNK inhibitor sequences, chimeric peptides, or nucleic acid sequences as defined herein, e.g. an JNK inhibitor sequence according to any of sequences of SEQ ID NOs: 1 to 4 and 13 to 20 and 33-100 and/or a chimeric peptide according to any of sequences of SEQ ID NOs: 9 to 12 and 23 to 32, and/or an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, may be utilized in treatment of dry eye syndrome e.g. after eye surgery or trauma, in particular after Lasik (laser-assisted in situ keratomileusis), commonly referred to simply as laser eye surgery.

The standard treatment of dry eye may involve the administration of artificial tears, cyclosporine (in particular cyclosporine A; e.g. Restasis®); autologous serum eye drops; lubricating tear ointments and/or the administration of (cortico-)steroids, for example in the form of drops or eye ointments. Therefore, the present invention also relates to the use of the JNK inhibitor sequences, chimeric peptides, or nucleic acid sequences as defined herein, e.g. an JNK inhibitor sequence according to any of sequences of SEQ ID NOs: 1 to 4 and 13 to 20 and 33-100 and/or a chimeric peptide according to any of sequences of SEQ ID NOs: 9 to 12 and 23 to 32, and/or an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, in a method of treatment of dry eye syndrome, wherein the method comprises the combined administration of the JNK inhibitor sequences, chimeric peptides, or nucleic acid sequences as defined herein together with a standard treatment for dry eye, in particular with any one of the above mentioned treatments. Particularly preferred is the combination with cyclosporine A and most preferably with artificial tears. Combined administration comprises the parallel administration and/or subsequent administration (either first the JNK inhibitor described herein and then the (cortico)steroids or vice versa). Certainly, subsequent and parallel administration may also be combined, e.g. the treatment is started with JNK inhibitors described herein and at a later point in time in the course of the treatment (cortico)steroids are given in parallel, or vice versa.

The suitable amount of the pharmaceutical composition to be used can be determined by routine experiments with animal models. Such models include, without implying any limitation, rabbit, sheep, mouse, rat, dog and non-human primate models. Preferred unit dose forms for injection include sterile solutions of water, physiological saline or mixtures thereof. The pH of such solutions should be adjusted to about 7.4. Suitable carriers for injection include hydrogels, devices for controlled or delayed release, polylactic acid and collagen matrices. Suitable pharmaceutically acceptable carriers for topical application include those, which are suitable for use in lotions, creams, gels and the like. If the compound is to be administered perorally, tablets, capsules and the like are the preferred unit dose form. The pharmaceutically acceptable carriers for the preparation of unit dose forms, which can be used for oral administration are well known in the prior art. The choice thereof will depend on secondary considerations such as taste, costs and storability, which are not critical for the purposes of the present invention, and can be made without difficulty by a person skilled in the art.

Pharmaceutical compositions for oral administration may be in tablet, capsule, powder or liquid form. A tablet may include a solid carrier as defined above, such as gelatin, and optionally an adjuvant. Liquid pharmaceutical compositions for oral administration generally may include a liquid carrier as defined above, such as water, petroleum, animal or vegetable oils, mineral oil or synthetic oil. Physiological saline solution, dextrose or other saccharide solution or glycols such as ethylene glycol, propylene glycol or polyethylene glycol may be included.

For example for intravenous, cutaneous or subcutaneous injection, injection at the site of affliction or in particular for instillation at the eye, the active ingredient will be preferably in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is a polypeptide, peptide, or nucleic acid molecule, other pharmaceutically useful compound according to the present invention that is to be given to an individual, administration is preferably in a “prophylactically effective amount or a “therapeutically effective amount” (as the case may be), this being sufficient to show benefit to the individual. The actual amount administered, and rate and time-course of administration, will depend on the nature and severity of what is being treated.

Prevention and/or treatment of a disease as defined herein typically includes administration of a pharmaceutical composition as defined above. The term “modulate” includes the suppression of expression of JNK when it is over-expressed in any of the above diseases. It also includes, without being limited thereto, suppression of phosphorylation of c-jun, ATF2 or NFAT4 in any of the above diseases, for example, by using at least one JNK inhibitor sequence according to any of sequences of SEQ ID NOs: 1 to 4 and 13 to 20 and 33-100 and/or at least one chimeric peptide according to any of sequences of SEQ ID NOs: 9 to 12 and 23 to 32, and/or at least one JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, as a competitive inhibitor of the natural c-jun, ATF2 and NFAT4 binding site in a cell. The term “modulate” also includes suppression of hetero- and homomeric complexes of transcription factors made up of, without being limited thereto, c-jun, ATF2, or NFAT4 and their related partners, such as for example the AP-1 complex that is made up of c-jun, AFT2 and c-fos. In some instances, “modulate” may then include the increase of JNK expression, for example by use of an IB peptide-specific antibody that blocks the binding of an IB-peptide to JNK, thus preventing JNK inhibition by the IB-related peptide.

Prevention and/or treatment of a subject with the pharmaceutical composition as disclosed above may be typically accomplished by administering (in vivo) an (“therapeutically effective”) amount of said pharmaceutical composition to a subject, wherein the subject may be e.g. any mammal, e.g. a human, a primate, mouse, rat, dog, cat, cow, horse or pig. The term “therapeutically effective” means that the active component of the pharmaceutical composition is of sufficient quantity to ameliorate the dry eye syndrome and/or associated symptoms.

Accordingly, peptides as defined above, e.g. at least one JNK inhibitor sequence according to any of sequences of SEQ ID NOs: 1 to 4 and 13 to 20 and 33-100 and/or at least one chimeric peptide according to any of sequences of SEQ ID NOs: 9 to 12 and 23 to 32, and/or at least one JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, may be utilized in a specific embodiment of the present invention to treat dry eye syndrome.

Peptides as defined above and as contained in the inventive pharmaceutical composition may be also encoded by nucleic acids. This is particularly advantageous, if the above peptides are administered for the purpose of gene therapy. In this context, gene therapy refers to therapy that is performed by administration of a specific nucleic acid as defined above to a subject, e.g. by way of a pharmaceutical composition as defined above, wherein the nucleic acid(s) exclusively comprise(s) L-amino acids. In this embodiment of the present invention, the nucleic acid produces its encoded peptide(s), which then serve(s) to exert a therapeutic effect by modulating function of the disease or disorder. Any of the methods relating to gene therapy available within the art may be used in the practice of the present invention (see e.g. Goldspiel, et al., 1993. Clin Pharm 12: 488-505).

In a preferred embodiment, the nucleic acid as defined above and as used for gene therapy is part of an expression vector encoding and expressing any one or more of the IB-related peptides as defined above within a suitable host, i.e. an JNK inhibitor sequence according to any of sequences of SEQ ID NOs: 1 to 4 and 13 to 20 and 33-100 and/or a chimeric peptide according to any of sequences of SEQ ID NOs: 9 to 12 and 23 to 32, and/or an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions. In a specific embodiment, such an expression vector possesses a promoter that is operably-linked to coding region(s) of a JNK inhibitor sequence. The promoter may be defined as above, e.g. inducible or constitutive, and, optionally, tissue-specific.

In another specific embodiment, a nucleic acid molecule as defined above is used for gene therapy, in which the coding sequences of the nucleic acid molecule (and any other desired sequences thereof) as defined above are flanked by regions that promote homologous recombination at a desired site within the genome, thus providing for intra-chromosomal expression of these nucleic acids (see e.g. Koller and Smithies, 1989. Proc Natl Acad Sci USA 86: 8932-8935).

Delivery of the nucleic acid as defined above according to the invention into a patient for the purpose of gene therapy, particular in the context of the above mentioned dry eye syndrome as defined above may be either direct (i.e. the patient is directly exposed to the nucleic acid or nucleic acid-containing vector) or indirect (i.e. cells are first transformed with the nucleic acid in vitro, then transplanted into the patient). These two approaches are known, respectively, as in vivo or ex vivo gene therapy. In a specific embodiment of the present invention, a nucleic acid is directly administered in vivo, where it is expressed to produce the encoded product. This may be accomplished by any of numerous methods known in the art including, e.g. constructing the nucleic acid as part of an appropriate nucleic acid expression vector and administering the same in a manner such that it becomes intracellular (e.g. by infection using a defective or attenuated retroviral or other viral vector; see U.S. Pat. No. 4,980,286); directly injecting naked DNA; using microparticle bombardment (e.g. a “GeneGun”; Biolistic, DuPont); coating the nucleic acids with lipids; using associated cell-surface receptors/transfecting agents; encapsulating in liposomes, microparticles, or microcapsules; administering it in linkage to a peptide that is known to enter the nucleus; or by administering it in linkage to a ligand predisposed to receptor-mediated endocytosis (see e.g. Wu and Wu, 1987. J Biol Chem 262: 4429-4432), which can be used to “target” cell types that specifically express the receptors of interest, etc.

An additional approach to gene therapy in the practice of the present invention involves transferring a nucleic acid as defined above into cells in in vitro tissue culture by such methods as electroporation, lipofection, calcium phosphate-mediated transfection, viral infection, or the like. Generally, the method of transfer includes the concomitant transfer of a selectable marker to the cells. The cells are then placed under selection pressure (e.g. antibiotic resistance) so as to facilitate the isolation of those cells that have taken up, and are expressing, the transferred gene. Those cells are then delivered to a patient. In a specific embodiment, prior to the in vivo administration of the resulting recombinant cell, the nucleic acid is introduced into a cell by any method known within the art including e.g. transfection, electroporation, microinjection, infection with a viral or bacteriophage vector containing the nucleic acid sequences of interest, cell fusion, chromosome-mediated gene transfer, microcell-mediated gene transfer, spheroplast fusion, and similar methods that ensure that the necessary developmental and physiological functions of the recipient cells are not disrupted by the transfer. See e.g. Loeffler and Behr, 1993. Meth Enzymol 217: 599-618. The chosen technique should provide for the stable transfer of the nucleic acid to the cell, such that the nucleic acid is expressible by the cell. Preferably, the transferred nucleic acid is heritable and expressible by the cell progeny.

In preferred embodiments of the present invention, the resulting recombinant cells may be delivered to a patient by various methods known within the art including, e.g. injection of epithelial cells (e.g. subcutaneously), application of recombinant skin cells as a skin graft onto the patient, and intravenous injection of recombinant blood cells (e.g. hematopoietic stem or progenitor cells). The total amount of cells that are envisioned for use depend upon the desired effect, patient state, and the like, and may be determined by one skilled within the art. Cells into which a nucleic acid can be introduced for purposes of gene therapy encompass any desired, available cell type, and may be xenogeneic, heterogeneic, syngeneic, or autogeneic. Cell types include, but are not limited to, differentiated cells such as epithelial cells, endothelial cells, keratinocytes, fibroblasts, muscle cells, hepatocytes and blood cells, or various stem or progenitor cells, in particular embryonic heart muscle cells, liver stem cells (International Patent Publication WO 94/08598), neural stem cells (Stemple and Anderson, 1992, Cell 71: 973-985), hematopoietic stem or progenitor cells, e.g. as obtained from bone marrow, umbilical cord blood, peripheral blood, fetal liver, and the like. In a preferred embodiment, the cells utilized for gene therapy are autologous to the patient.

Alternatively and/or additionally, for treating diseases as mentioned herein targeting therapies may be used to deliver the JNK inhibitor sequences, chimeric peptides, and/or nucleic acids as defined above more specifically to certain types of cell, by the use of targeting systems such as (a targeting) antibody or cell specific ligands. Antibodies used for targeting are typically specific for cell surface proteins of cells associated with any of the diseases as defined below. By way of example, these antibodies may be directed to cell surface antibodies such as e.g. B cell-associated surface proteins such as MHC class II DR protein, CD18 (LFA-1 beta chain), CD45RO, CD40 or Bgp95, or cell surface proteins selected from e.g. CD2, CD2, CD4, CD5, CD7, CD8, CD9, CD10, CD13, CD16, CD19, CD20, CD21, CD22, CD23, CD24, CD25, CD30, CD33, CD34, CD38, CD39, CD4, CD43, CD45, CD52, CD56, CD68, CD71, CD138, etc. Targeting constructs may be typically prepared by covalently binding the JNK inhibitor sequences, chimeric peptides, and nucleic acids as defined herein according to the invention to an antibody specific for a cell surface protein or by binding to a cell specific ligand. Proteins may e.g. be bound to such an antibody or may be attached thereto by a peptide bond or by chemical coupling, crosslinking, etc. The targeting therapy may then be carried out by administering the targeting construct in a pharmaceutically efficient amount to a patient by any of the administration routes as defined below, e.g. intraperitoneal, nasal, intravenous, oral and patch delivery routes. Preferably, the JNK inhibitor sequences, chimeric peptides, or nucleic acids as defined herein according to the invention, being attached to the targeting antibodies or cell specific ligands as defined above, may be released in vitro or in vivo, e.g. by hydrolysis of the covalent bond, by peptidases or by any other suitable method. Alternatively, if the JNK inhibitor sequences, chimeric peptides, or nucleic acids as defined herein according to the invention are attached to a small cell specific ligand, release of the ligand may not be carried out. If present at the cell surface, the chimeric peptides may enter the cell upon the activity of its trafficking sequence. Targeting may be desirable for a variety of reasons; for example if the JNK inhibitor sequences, chimeric peptides, and nucleic acids as defined herein according to the invention are unacceptably toxic or if it would otherwise require a too high dosage.

Instead of administering the JNK inhibitor sequences and/or chimeric peptides as defined herein according to the invention directly, they could be produced in the target cells by expression from an encoding gene introduced into the cells, e.g. from a viral vector to be administered. The viral vector typically encodes the JNK inhibitor sequences and/or chimeric peptides as defined herein according to the invention. The vector could be targeted to the specific cells to be treated. Moreover, the vector could contain regulatory elements, which are switched on more or less selectively by the target cells upon defined regulation. This technique represents a variant of the VDEPT technique (virus-directed enzyme prodrug therapy), which utilizes mature proteins instead of their precursor forms.

Alternatively, the JNK inhibitor sequences and/or chimeric peptides as defined herein could be administered in a precursor form by use of an antibody or a virus. These JNK inhibitor sequences and/or chimeric peptides may then be converted into the active form by an activating agent produced in, or targeted to, the cells to be treated. This type of approach is sometimes known as ADEPT (antibody-directed enzyme prodrug therapy) or VDEPT (virus-directed enzyme prodrug therapy); the former involving targeting the activating agent to the cells by conjugation to a cell-specific antibody, while the latter involves producing the activating agent, e.g. a JNK inhibitor sequence or the chimeric peptide, in a vector by expression from encoding DNA in a viral vector (see for example, EP-A-415731 and WO 90/07936).

According to a further embodiment, the JNK inhibitor sequences, chimeric peptides, or nucleic acid sequences as defined herein, e.g. an JNK inhibitor sequence according to any of sequences of SEQ ID NOs: 1 to 4 and 13 to 20 and 33-100 and/or a chimeric peptide according to any of sequences of SEQ ID NOs: 9 to 12 and 23 to 32, and/or an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or variants or fragments thereof within the above definitions, may be utilized in (in vitro) assays (e.g. immunoassays) to detect, prognose, diagnose, or monitor dry eye syndrome as defined above, or monitor the treatment thereof. The immunoassay may be performed by a method comprising contacting a sample derived from a patient with an antibody to an JNK inhibitor sequence, a chimeric peptide, or a nucleic acid sequence, as defined above, under conditions such that immunospecific-binding may occur, and subsequently detecting or measuring the amount of any immunospecific-binding by the antibody. In a specific embodiment, an antibody specific for an JNK inhibitor sequence, a chimeric peptide or a nucleic acid sequence may be used to analyze a tissue or serum sample from a patient for the presence of JNK or a JNK inhibitor sequence; wherein an aberrant level of JNK is indicative of a diseased condition. The immunoassays that may be utilized include, but are not limited to, competitive and non-competitive assay systems using techniques such as Western Blots, radioimmunoassays (RIA), enzyme linked immunosorbent assay (ELISA), “sandwich” immunoassays, immunoprecipitation assays, precipitin reactions, gel diffusion precipitin reactions, immunodiffusion assays, agglutination assays, fluorescent immunoassays, complement-fixation assays, immunoradiometric assays, and protein-A immunoassays, etc. Alternatively, (in vitro) assays may be performed by delivering the JNK inhibitor sequences, chimeric peptides, nucleic acid sequences or antibodies to JNK inhibitor sequences or to chimeric peptides, as defined above, to target cells typically selected from e.g. cultured animal cells, human cells or micro-organisms, and to monitor the cell response by biophysical methods typically known to a skilled person. The target cells typically used therein may be cultured cells (in vitro) or in vivo cells, i.e. cells composing the organs or tissues of living animals or humans, or microorganisms found in living animals or humans.

The present invention additionally provides the use of kits for diagnostic or therapeutic purposes, particular for the treatment, prevention or monitoring of dry eye syndrome as defined above, wherein the kit includes one or more containers containing JNK inhibitor sequences, chimeric peptides, nucleic acid sequences and/or antibodies to these JNK inhibitor sequences or to chimeric peptides as defined above, e.g. an anti-JNK inhibitor sequence antibody to an JNK inhibitor sequence according to any of sequences of SEQ ID NOs: 1 to 4 and 13 to 20 and 33-100, to a chimeric peptide according to any of sequences of SEQ ID NOs: 9 to 12 and 23 to 32, to an JNK inhibitor sequence according to any of sequences of SEQ ID NOs: 1 to 4 and 13 to 20 and 33-100 comprising a trafficking sequence according to any of SEQ ID NOs: 5 to 8 and 21 to 22, or to or variants or fragments thereof within the above definitions, or such an anti-JNK inhibitor sequence antibody and, optionally, a labeled binding partner to the antibody. The label incorporated thereby into the antibody may include, but is not limited to, a chemiluminescent, enzymatic, fluorescent, colorimetric or radioactive moiety. In another specific embodiment, kits for diagnostic use in the treatment, prevention or monitoring of dry eye syndrome are provided which comprise one or more containers containing nucleic acids that encode, or alternatively, that are the complement to, an JNK inhibitor sequence and/or a chimeric peptide as defined above, optionally, a labeled binding partner to these nucleic acids, are also provided. In an alternative specific embodiment, the kit may be used for the above purposes as a kit, comprising one or more containers, a pair of oligonucleotide primers (e.g. each 6-30 nucleotides in length) that are capable of acting as amplification primers for polymerase chain reaction (PCR; see e.g. Innis, et al., 1990. PCR PROTOCOLS, Academic Press, Inc., San Diego, Calif.), ligase chain reaction, cyclic probe reaction, and the like, or other methods known within the art used in context with the nucleic acids as defined above. The kit may, optionally, further comprise a predetermined amount of a purified JNK inhibitor sequence as defined above, a chimeric peptide as defined above, or nucleic acids encoding these, for use as a diagnostic, standard, or control in the assays for the above purposes.

The present invention is not to be limited in scope by the specific embodiments described herein. Indeed, various modifications of the invention in addition to those described herein will become apparent to those skilled in the art from the foregoing description and accompanying figures. Such modifications fall within the scope of the appended claims.

Various publications are cited herein, the disclosures of which are incorporated by reference in their entirety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

### DESCRIPTION OF FIGURES

FIG. 1 shows the IB1 cDNA sequence from rat and its predicted amino acid sequence (SEQ ID NO:102).

FIG. 2 shows the IB1 protein sequence from rat encoded by the exon-intron boundary of the rIB1 gene—splice donor (SEQ ID NO:103).

FIG. 3 shows the IB1 protein sequence from Homo sapiens (SEQ ID NO:104).

FIG. 4 shows the IB1 cDNA sequence from Homo sapiens (SEQ ID NO:105).

FIG. 5 shows the mean calculated TBUT AUC values for animals with scopolamine induced dry eye syndrome. Shown are the results for animals treated with vehicle, and 3 different concentrations of an all-D-retro-inverso JNK-inhibitor (poly-)peptide with the sequence of SEQ ID NO: 11.

FIG. 6 shows the mean calculated PRTT AUCs for animals with scopolamine induced Dry Eye (Day 7-21). Shown are the results for animals treated with vehicle, and 3 different concentrations of an all-D-retro-inverso JNK-inhibitor (poly-)peptide with the sequence of SEQ ID NO: 11.

FIG. 7 shows the mean histological Cornea Lesion Scores for animals with scopolamine induced dry eye syndrome. Shown are the results for animals treated with vehicle, and 3 different concentrations of an all-D-retro-inverso JNK-inhibitor (poly-)peptide with the sequence of SEQ ID NO: 11.

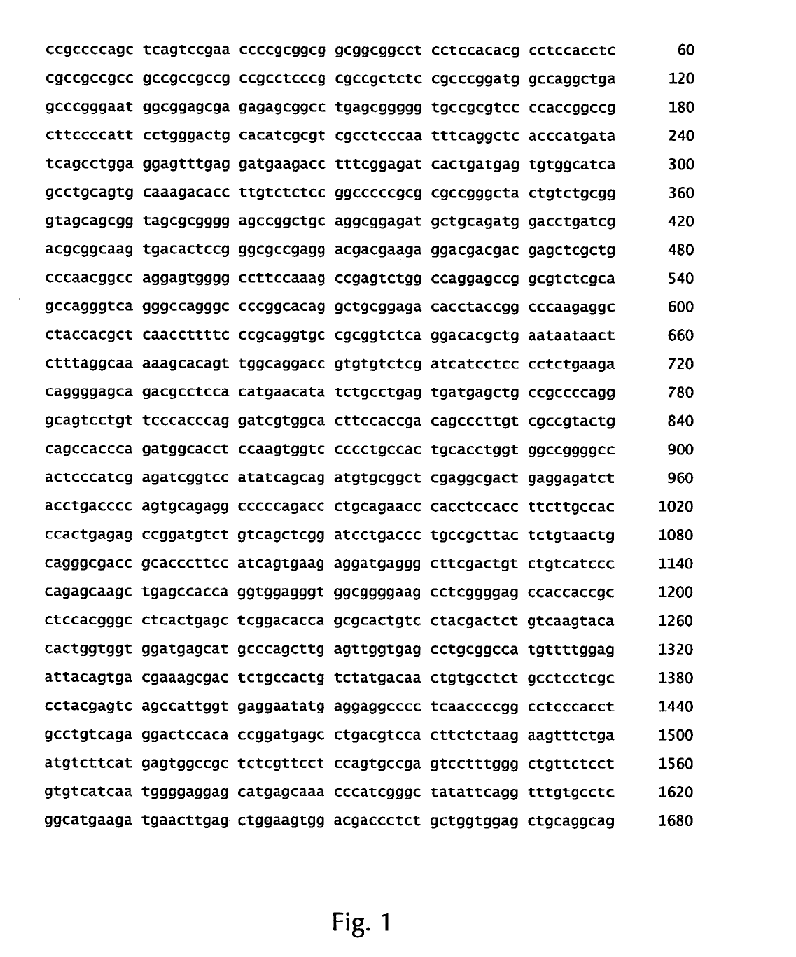

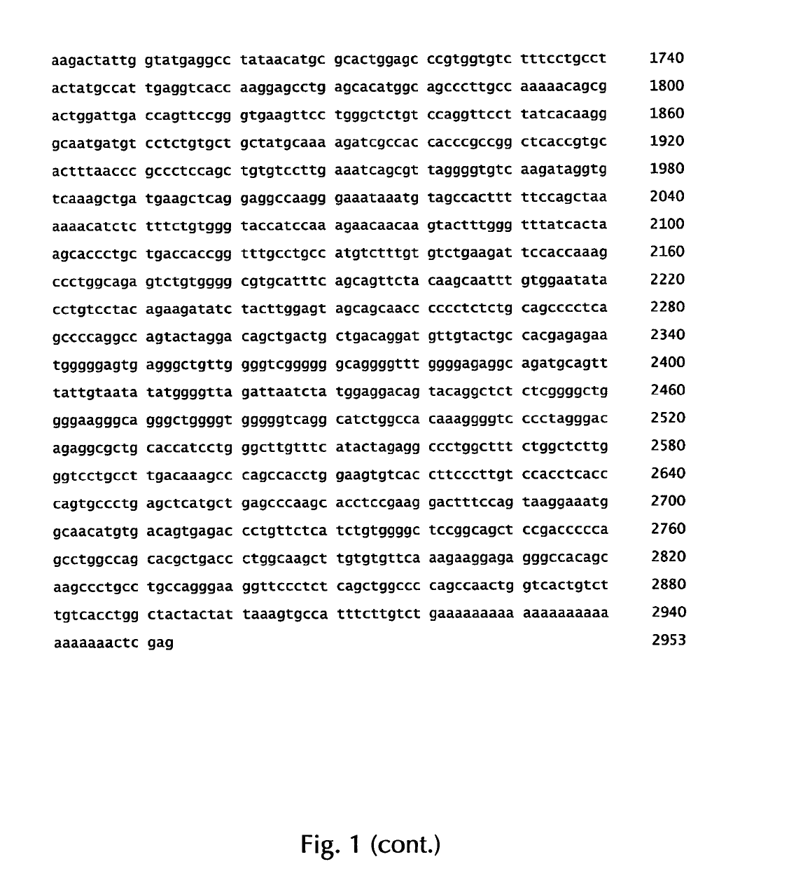

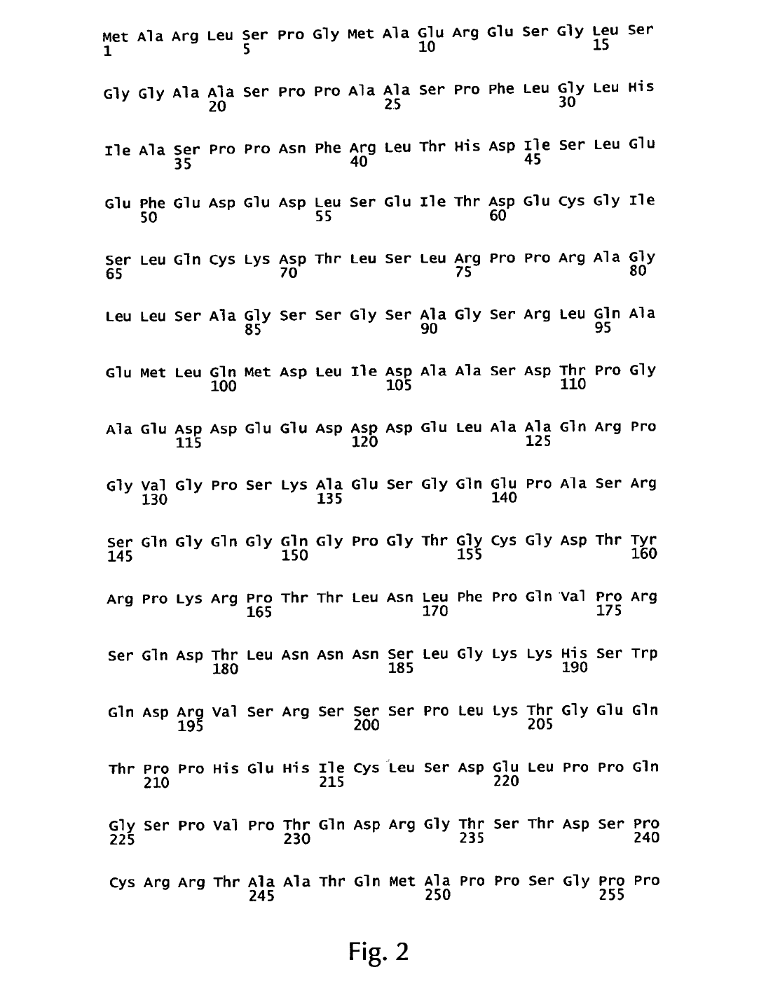

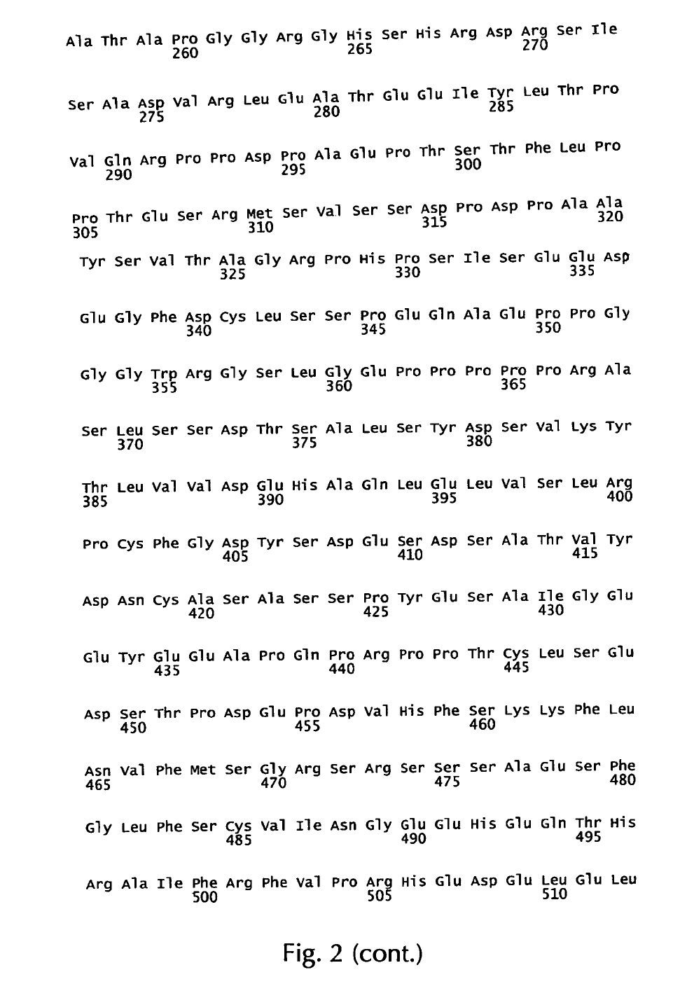

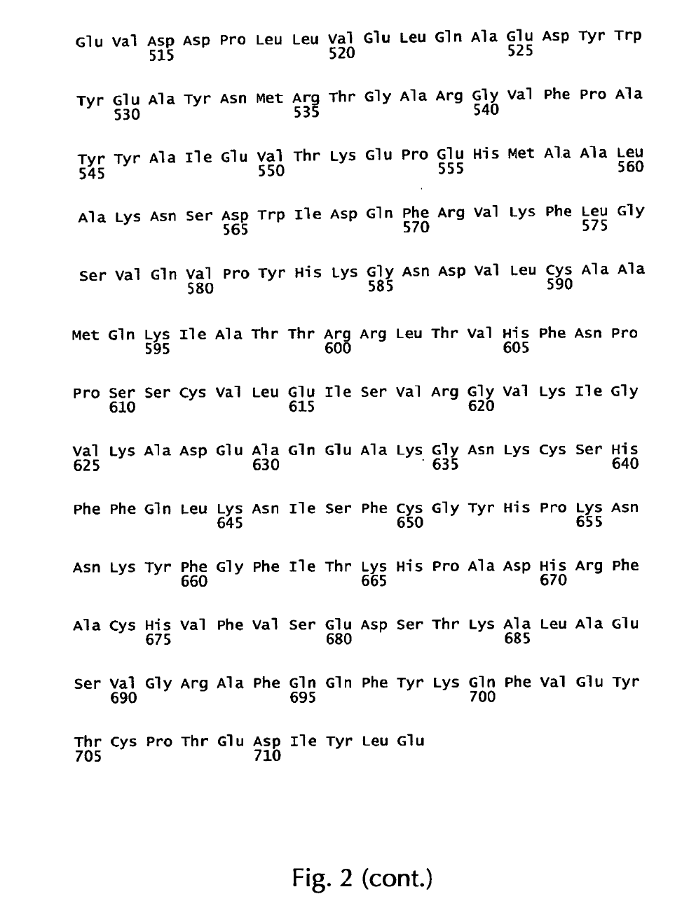

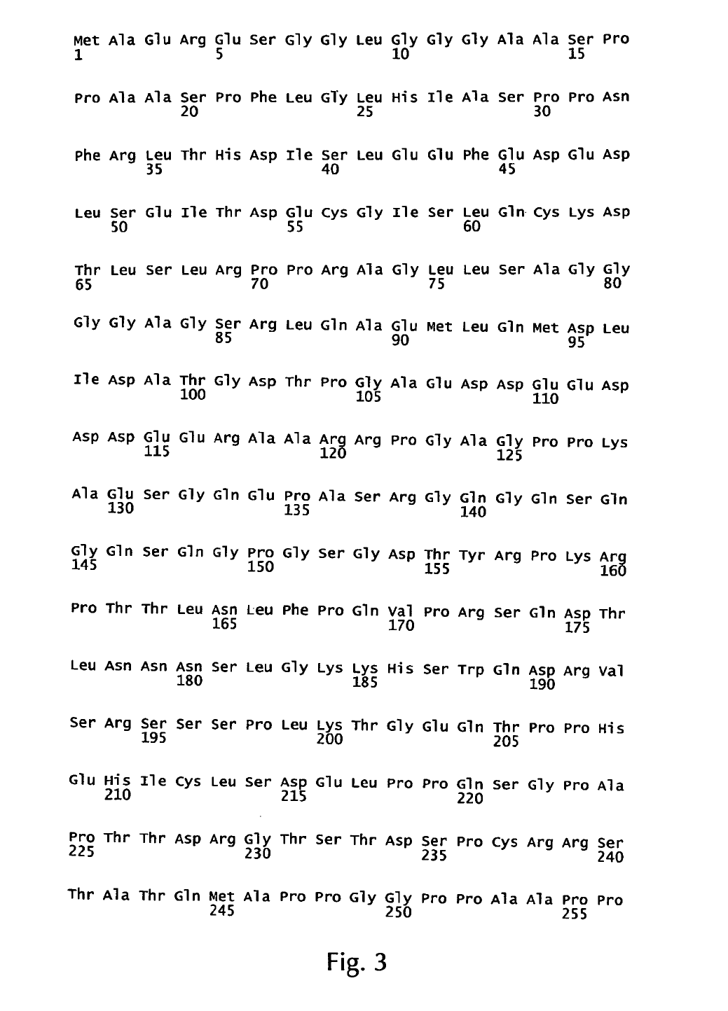

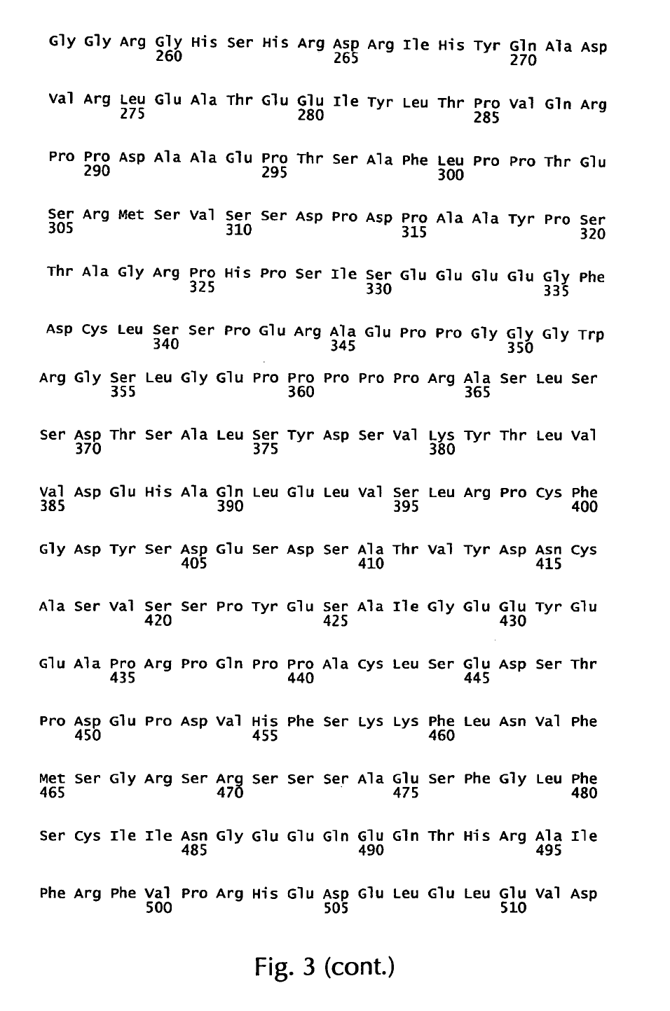

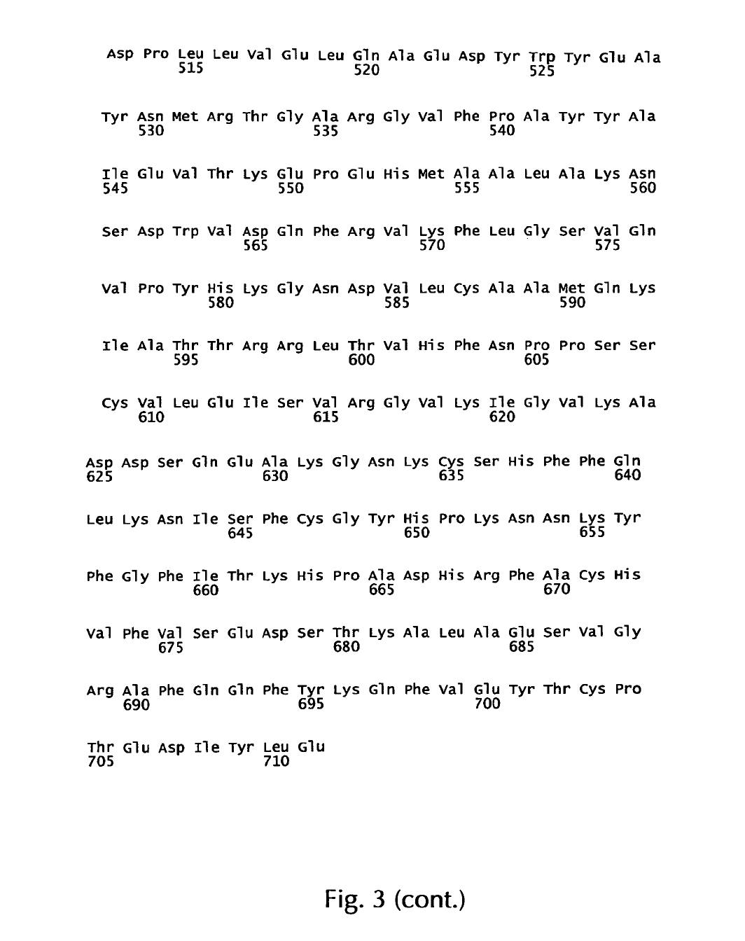

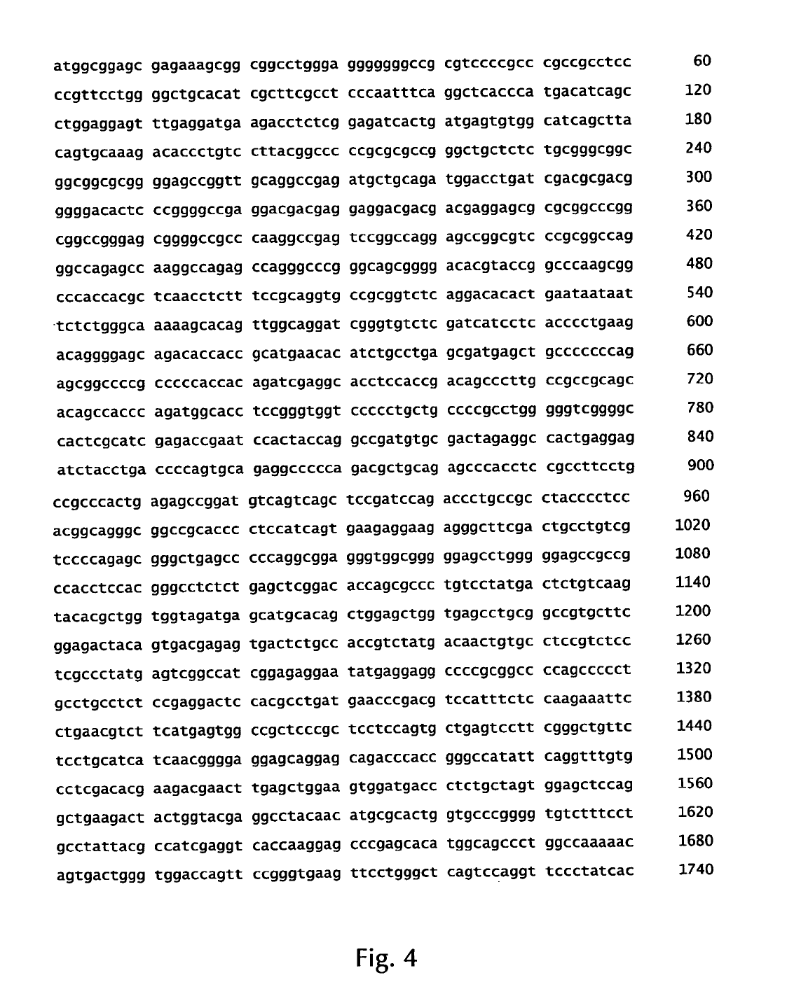

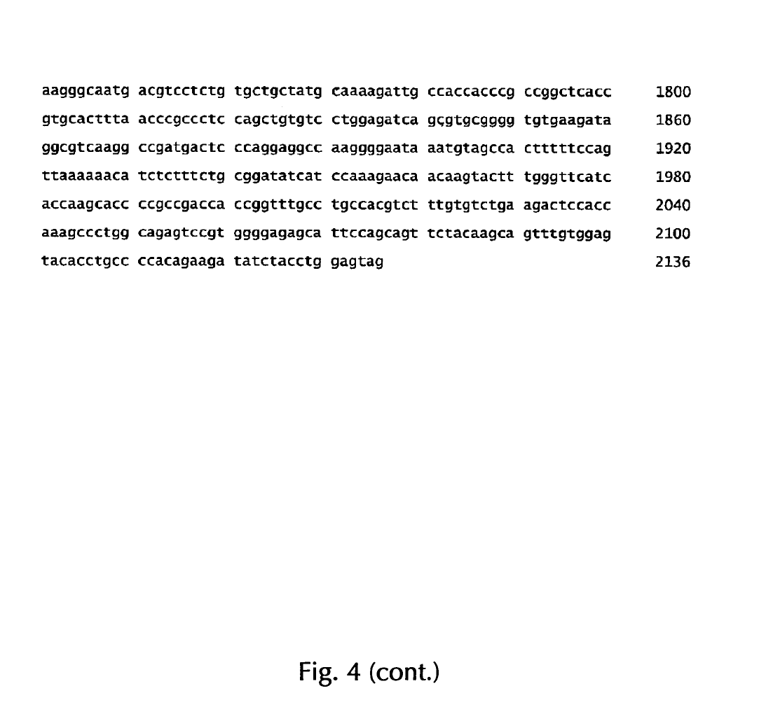

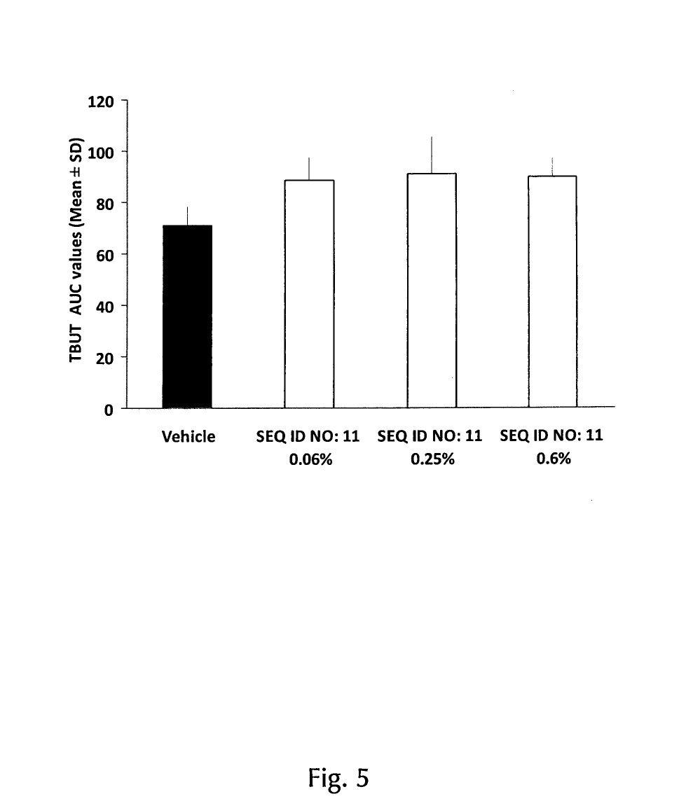

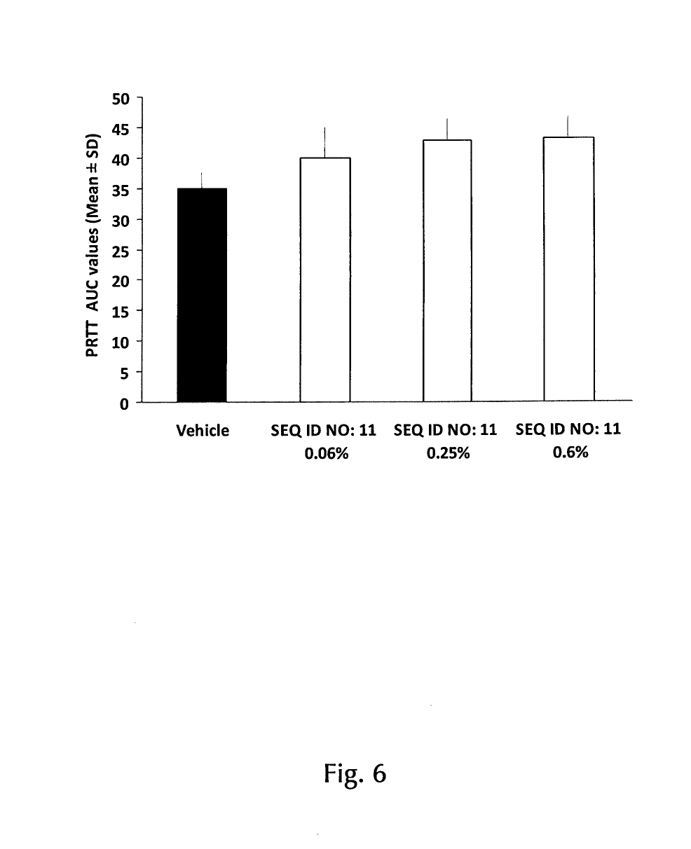

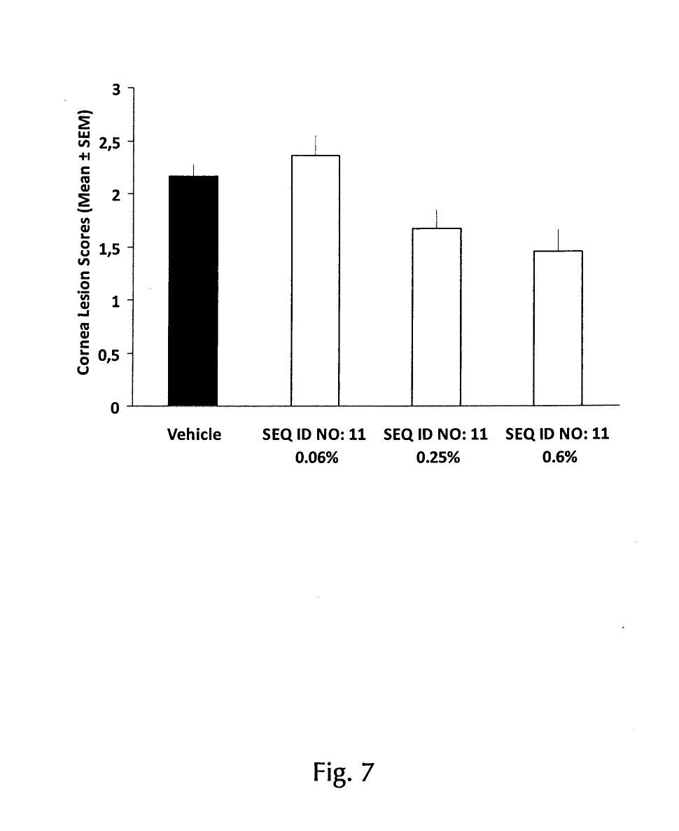

### EXAMPLES

### Example 1

### Solutions and Products

An all-D-retro-inverso JNK-inhibitor (poly-)peptide of SEQ ID NO: 11 was produced by Polypeptide Laboratories (France) and purified by High Performance Liquid Chromatography (HPLC). It was analyzed by mass spectrometry for identity and RP-HPLC for purity (Polypeptide Laboratories, France). Once lyophilized, the powder was stored at 2-8° C.

### Example 2

### Effect of the All-D-Retro-Inverso JNK-Inhibitor (Poly-)Peptide of SEQ ID NO: 11 at Three Doses in a Scopolamine-Induced Model of Dry Eye in Mice

### Study Concept

The objective of this study was to assess the effects of the all-D-retro-inverso JNK-inhibitor (poly-)peptide of SEQ ID NO: 11 at three dose levels in a mouse model of scopolamine-induced dry eye.

The peptide of SEQ ID NO: 11 was tested for efficacy in this murine model of dry eye. The peptide was tested at a low, medium and a high dose. For the peptide of SEQ ID NO: 11 the concentrations measured in the formulation samples for low, medium and high dose levels were 0.06% (w/v), 0.25% (w/v) and 0.6% (w/v), respectively. The vehicle, which also served as the negative control, was 0.9% Sodium Chloride for Injection USP.

The study consisted of a total of 5 groups of female C57BL/6 mice, comprising 4 groups of 12 mice each and an additional group of 4 mice. Bilateral short-term dry eye was induced by a combination of scopolamine hydrobromide (Sigma-Aldrich Corp., St. Louis, Mo.) injection (subcutaneous (SC), four times daily, 0.5 mg/dose, Days 0-21) and by exposing mice to the drying environment of constant air draft. Starting on Day 1, mice of Groups 1-4 were treated three times daily (TID) for 21 days with bilateral topical ocular (oculus uterque; OU) administration (5 μL/eye/dose) of vehicle (0.9% sterile saline; negative control article); or the peptide of SEQ ID NO: 11 (0.06%, 0.25% and 0.6%). Mice of Group 5 were maintained as un-induced, (no dry eye) untreated controls.

During the in-life (treatment) period, clinical observations were recorded once daily; slit-lamp examination (SLE) with corneal fluorescein staining, tear break-up time test (TBUT), and phenol red thread test (PRTT) were performed three times per week. Necropsies were performed on Day 22; eyes, eye lids, conjunctivae, and lacrimal glands were collected from both eyes of each animal. Tissues from the right eyes (oculus dexter, OD) were fixed and then evaluated microscopically. Tissues from the left eyes (oculus sinister; OS) were flash-frozen in liquid nitrogen and stored frozen at −80° C. for possible subsequent analyses.

TABLE 3
Experimental Design

| | | | Treatment |
|---|---|---|---|
| | Number | Induction of | (TID, OU, |
| | of | Dry Eye | 5 μL/eye) |
| | animals | (QID, SC) | Days 1* to |
| Group | (females) | Days 0 to 21 | 21 |
| 1 | 12 | Scopolamine | Vehicle |
| 2 | 12 | (200 μL of | peptide of |
| | | 2.5 mg/mL | SEQ ID NO: |
| | | sol., 0.5 | 11 |
| | | mg/dose) | (0.06%) |
| 3 | 12 | | peptide of |
| | | | SEQ ID NO: |
| | | | 11 |
| | | | (0.25%) |
| 4 | 12 | | peptide of |
| | | | SEQ ID NO: |
| | | | 11 |
| | | | (0.6%) |
| 5 | 4 | No dry eye | No |
| | | induction | treatment |

### Methods

### 1. Dose Preparation

The (poly-)peptide of SEQ ID NO: 11 was obtained from Polypeptide Laboratories (France) as a 1.5-mL clear plastic microfuge vial containing 300.65 mg of dry powder.

Prior to the start of the study, the (poly-)peptide of SEQ ID NO: 11 was formulated in sterile saline (vehicle). Dosing solutions at each concentration were sterilized using 0.2-μm filters, aliquoted to multiple pre-labeled vials, and frozen at −20° C. The concentrations measured in the formulation samples were 0.058%, 0.25% and 0.624% rounded to 0.06%, 0.25% and 0.6%.

On each day of dosing, one set of dosing solutions was thawed and used for that day's dose administrations. The control (vehicle) was provided ready to dose; no dose preparation was necessary.

### 2. Slit-Lamp Examinations (SLE)

Prior to entry into the study, each animal underwent a SLE and indirect ophthalmic examination using topically-applied fluorescein. Ocular findings were recorded using the Draize scale ocular scoring. SLE and Draize scoring were repeated three times a week during the in-life period.

### 3. Tear Break-Up Time (TBUT) Test and Subsequent Corneal Examination

The TBUT test was conducted three times weekly by measuring the time elapsed in seconds between a complete blink after application of fluorescein to the cornea and the appearance of the first random dry spot in the tear film. To perform the TBUT, 0.1% liquid sodium fluorescein was dropped into the conjunctival sac, the eyelids were manually closed three times and then held open revealing a continuous fluorescein-containing tear film covering the cornea, and the time (in seconds) required for the film to break (appearance of a dry spot or streak) was recorded. At least ninety seconds later, corneal epithelial damage was graded using a slit-lamp with a cobalt blue filter after another drop of 0.1% fluorescein was reapplied to the cornea; the cornea then was scored per the Draize ocular scale.

### 4. Phenol Red Thread Tear Test (PRTT)

Tear production was measured three times a week in both eyes using PRTT test strips (Zone-Quick; Menicon, Nagoya, Japan). Prior to the first treatment of the day, a thread was applied to the lateral canthus of the conjunctival fornix of each eye for 30 seconds under slit-lamp biomicroscopy. Tear migration up the tread (i.e., the length of the wetted cotton thread) was measured using a millimeter scale.

### 5. Necropsy and Pathology

At necropsy on Day 22, both eyes from each animal, including the globes, lacrimal glands, eyelids, and conjunctivae, were excised. The right eye and associated tissues were fixed by overnight submersion in modified Davidson's solution followed by transfer to 10% neutral buffered formalin (NBF). The fixed tissues of the right eye were dehydrated, embedded in paraffin, sectioned at 3 to 5-μm thicknesses, and slide-mounted tissues were stained with hematoxylin and eosin (H & E). Stained slides were evaluated via light microscopy. Detailed and complete histopathologic assessment was conducted on all parts of the eye, with at least two section levels being examined histopathologically for each right eye. Special attention was paid to the cornea, epithelia (including goblet cells) of the conjunctiva and cornea, as well as the lacrimal gland. These tissues were scored for injury based upon a 0-4 scale, with 0 being normal, 1 being minimal, 2 being mild, 3 being moderate, and 4 being severe. For each cornea, scores were based on corneal epithelium thickness, and corneal inflammation. Conjunctivae were scored for erosion and inflammation as well as presence or absence of goblet cells.

### Results

Four-times daily SC administration of scopolamine (0.5 mg/dose) induced a dry eye syndrome in female C57BL/6 mice characterized by a decrease in the volume of aqueous tear production and changes in the physiochemical properties of the tears rendering them less capable of maintaining a stable tear film able to effectively lubricate and protect the eye.

### 1. Tear Break-Up Time (TBUT) Teat and Corneal Examination

The tear break-up time tests (TBUTs) were performed prior to the induction of dry eye, and again on Days 2, 4, 7, 9, 11, 14, 16, 18 and 21 after dry eye induction. After initiation of dosing with scopolamine (dry eye induction) TBUT mean values began to decrease in all animals. TBUT means for animals treated with mid and high-dose of the peptide of SEQ ID NO: 11, Groups 3 and 4, continued to decline after onset of dosing, reaching a nadir on Day 9, while the low-dose Group 2 increased on Day 9. The low, medium and high-dose TBUT means (Groups 2, 3 and 4, respectively) were above the vehicle group. Groups treated with low, mid and high dose levels of peptide of SEQ ID NO: 11 (Groups 2-4) showed generally dose-dependent increases in TBUT.

TABLE 4
Mean Calculated
TBUT AUC Values:

| | TBUT |
|---|---|
| Group | AUC |
| Group 1 | 71.19 |
| Group 2 | 88.54 |
| Group 3 | 91.19 |
| Group 4 | 89.98 |
| Group 5 | 124.54 |

### 2. Phenol Red Thread Tear Test (PRTT)

PRTT tests were performed prior to the induction of dry eye, and again on Days 2, 4, 7, 9, 11, 14, 16, 18 and 21. PRTT values from Day 0 to Day 4 decreased in all mice that had dry eye induced, indicating a decrease in tear production after the administration of scopolamine and exposure to a drying environment of increased air draft created by the blowers. The nadir in PRTT in most groups occurred at approximately Day 7. PRTT kept decreasing in the vehicle control group (Group 1) reaching a nadir on Day 14. After the nadir, there was an increase in all dry eye groups. These findings indicate that initiation of scopolamine treatment one day earlier than initiation of compound treatment was sufficient to initiate physiological changes in the eye associated with dry eye syndrome.

Groups treated with low, mid and high dose levels of the peptide of SEQ ID NO: 11 (0.06%, 0.25% and 0.6%, Groups 2, 3 and 4, respectively) showed generally dose-dependent increases in PRTT.

TABLE 5
Mean PRTT
AUC Values

| Group | PRTT AUC |
|---|---|
| Group 1 | 35.02 |
| Group 2 | 39.96 |
| Group 3 | 42.79 |
| Group 4 | 43.17 |
| Group 5 | 113.63 |

### 3. Histopathology

In this study histologic changes were generally confined to the cornea. Findings in the cornea consisted of increased keratinization of the corneal epithelial surface, increased thickness of the corneal epithelium, increased cellularity of the corneal epithelium, mildly increased incidence of mitosis of the basal epithelial layer consistent with increased epithelial cell turnover. These findings are indicative of a physiologic adaptive response to corneal drying and corneal surface irritation. Surface ulceration, corneal stromal edema and inflammatory infiltrate into the cornea were not seen in this study. The eyes in Group 5, the untreated group (normal mice, no scopolamine treatment), were within normal limits. There was some minimal nonsuppurative inflammation of the eye lids scattered throughout all groups, but the conjunctiva, retina, lacrimal glands and other parts of the eye were within normal limits. Goblet cells appeared to be within limits in all groups. Goblet cells are a primary producer of mucin which helps the tears form a stronger more adhesive film.

Mild to moderate corneal changes were noted in all groups except the untreated normal eye group (Group 5) and were slightly more severe in Group 1, the vehicle-treated group and Group 2, the low-dose of the peptide of SEQ ID NO: 11, in comparison to the other treatment groups. These findings were consistent with the positive beneficial effects of increased tear production on the cornea.

The high-dose of peptide of SEQ ID NO: 11 was the most effective in reducing/ameliorating the corneal changes associated with this murine dry eye model.

1. Use of a JNK inhibitor (poly-)peptide comprising less than 150 amino acids in length for the preparation of a pharmaceutical composition for treating dry eye syndrome.

2. The use of a JNK inhibitor (poly-)peptide according to claim 1, wherein the JNK inhibitor (poly-)peptide comprises a range of 5 to 150 amino acid residues, more preferably 10 to 100 amino acid residues, even more preferably 10 to 75 amino acid residues and most preferably a range of 10 to 50 amino acid residues.

3. The use of a JNK inhibitor (poly-)peptide of claim 1, wherein the JNK inhibitor (poly-)peptide binds c-jun amino terminal kinase QNK).

4. The use of a JNK inhibitor (poly-)peptide of claim 1, wherein the JNK inhibitor (poly-)peptide inhibits the activation of at least one JNK targeted transcription factor when the JNK inhibitor (poly-)peptide is present in a JNK expressing cell.

5. The use of a JNK inhibitor (poly-)peptide of claim 4, wherein the JNK targeted transcription factor is selected from the group consisting of c-Jun, ATF2, and ElkI.

6. The use of a JNK inhibitor (poly-)peptide of claim 1, wherein the JNK inhibitor (poly-)peptide alters a JNK effect when the peptide is present in a JNK expressing cell.

7. The use of a JNK inhibitor sequence of claim 1, wherein the JNK inhibitor (poly-)peptide is composed of L-amino acids, D-amino acids, or a combination of both, preferably comprises at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the JNK inhibitor (poly-)peptide in a blockwise, a non-blockwise or in an alternate manner.

8. The use according to claim 1, wherein the JNK inhibitor (poly-)peptide comprises a fragment, variant, or variant of such fragment of a human or rat IB1 (poly-) peptide as defined or encoded by any of sequences according to SEQ ID NO: 102, SEQ ID NO: 103, SEQ ID NO: 104 or SEQ ID NO: 105.

9. The use of a JNK inhibitor (poly-)peptide according to claim 1, wherein the inhibitor sequence comprises or consists of at least one amino acid sequence according to SEQ ID NOs: 1 to 4, 13 to 20 and 33 to 100, or a fragment, derivative or variant thereof.

10. Use of a chimeric (poly-)peptide comprising at least one first domain and at least one second domain linked by a covalent bond, the first domain comprising a trafficking (poly-)peptide, and the second domain comprising a JNK inhibitor (poly-)peptide as defined in claim 1 for the preparation of a pharmaceutical composition for treating dry eye syndrome.

11. The use of the chimeric (poly-)peptide of claim 10, wherein the chimeric (poly)peptide is composed of L-amino acids, D-amino acids, or a combination of both, preferably comprises at least 1 or even 2, preferably at least 3, 4 or 5, more preferably at least 6, 7, 8 or 9 and even more preferably at least 10 or more D- and/or L-amino acids, wherein the D- and/or L-amino acids may be arranged in the chimeric peptide in a blockwise, a non-blockwise or in an alternate manner.

12. The use of the chimeric (poly-)peptide of claim 10, wherein the trafficking (poly-)peptide comprises the amino acid sequence of a human immunodeficiency virus TAT polypeptide.

13. The use of the chimeric (poly-)peptide of claim 10, wherein the trafficking sequence consists of or comprises the amino acid sequence of SEQ ID NO: 5, 6, 7, 8, 21 or 22.

14. The use of the chimeric (poly-)peptide of claim 10, wherein the trafficking (poly-)peptide augments cellular uptake of the peptide.

15. The use of the chimeric (poly-)peptide of claim 10, wherein the trafficking (poly-)peptide directs nuclear localization of the peptide.

16. The use of the chimeric (poly-)peptide of claim 10, wherein the chimeric (poly-)peptide consists of or comprises the amino acid sequence of any of SEQ ID NOs: 9 to 12 and 23 to 32, or a fragment, or variant thereof.

17. The use of the chimeric peptide of claim 10, wherein the chimeric (poly-)peptide consists of or comprises the amino acid sequence of SEQ ID NO: 9 or 11.

18. Use of an isolated nucleic acid encoding a JNK inhibitor (poly-)peptide or a chimeric (poly-)peptide for the preparation of a pharmaceutical composition for treating dry eye syndrome, wherein:
the JNK inhibitor (poly-)peptide comprises less than 150 amino acids in length for the preparation of a pharmaceutical composition for treating dry eye syndrome; and
the chimeric (poly-)peptide comprises at least one first domain and at least one second domain linked by a covalent bond, the first domain comprising a trafficking (poly-)peptide, and the second domain comprising the JNK inhibitor (poly-)peptide.

19. Use of a vector comprising the nucleic acid as defined in claim 18 for the preparation of a pharmaceutical composition for treating dry eye syndrome.

20. Use of a cell comprising the vector as defined in claim 19 for the preparation of a pharmaceutical composition for treating dry eye syndrome.

21. Use according to claim 1, wherein the pharmaceutical composition is to be administered by an administration route selected from the group consisting of parenteral routes, including intravenous, intramuscular, subcutaneous, intradermal, transdermal, enteral routes, including orally, rectally, topical routes, including nasal, intranasal, other routes, including epidermal or patch delivery, and local administration to the eye, in particular intravitreous administration, subconjunctival administration and/or instillation.

22. The use according to claim 1, wherein a dose (per kg bodyweight) of the JNK inhibitor (poly-)peptide and/or chimeric (poly-)peptide is in the range of up to 10 mmol/kg, preferably up to 1 mmol/kg, more preferably up to 100 pmol/kg, even more preferably up to 10 pmol/kg, even more preferably up to 1 pmol/kg, even more preferably up to 100 nmol/kg, most preferably up to 50 nmol/kg.

23. The use according to claim 1, wherein a dose of the JNK inhibitor (poly-)peptide and/or chimeric (poly-)peptide is in the range of from about 1 pmol/kg to about 1 mmol/kg, from about 10 pmol/kg to about 0.1 mmol/kg, from about 10 pmol/kg to about 0.01 mmol/kg, from about 50 pmol/kg to about 1 pmol/kg, from about 100 pmol/kg to about 500 nmol/kg, from about 200 pmol/kg to about 300 nmol/kg, from about 300 pmol/kg to about 100 nmol/kg, from about 500 pmol/kg to about 50 nmol/kg, from about 750 pmol/kg to about 30 nmol/kg, from about 250 pmol/kg to about 5 nmol/kg, from about 1 nmol/kg to about 10 nmol/kg, or a combination of any two of said values.

24. The use according to claim 1, wherein the JNK inhibitor (poly-)peptide consists of the sequence of SEQ ID NO:11 and is preferably administered by way of instillation.


SEQUENCE LISTING (105 sequences)

SEQ ID NO: 1 - protein, 19 aa, Artificial
  Description of sequence Peptide L-IB1(s) (see Table 1)
RPKRPTTLNLFPQVPRSQD

SEQ ID NO: 2 - protein, 19 aa, Artificial
  Description of sequence Peptide D-IB1(s) (see Table 1)
DQSRPVQPFLNLTTPRKPR

SEQ ID NO: 3 - protein, 19 aa, Artificial
  Description of sequence Peptide L-IB (generic) (s) (see Table 1)
XXRPTTLXLXXXXXXXQDX

SEQ ID NO: 4 - protein, 19 aa, Artificial
  Description of sequence Peptide D-IB (generic) (s) (see Table 1)
XDQXXXXXXXLXLTTPRXX

SEQ ID NO: 5 - protein, 10 aa, Artificial
  Description of sequence  Peptide L-TAT (see Table 1)
GRKKRRQRRR

SEQ ID NO: 6 - protein, 10 aa, Artificial
  Description of sequence Peptide D-TAT (see Table 1)
RRRQRRKKRG

SEQ ID NO: 7 - protein, 11 aa, Artificial
  Description of sequence Peptide L-generic-TAT (s) (see Table 1)
XRKKRRQRRRX

SEQ ID NO: 8 - protein, 11 aa, Artificial
  Description of sequence Peptide D-generic-TAT (s) (see Table 1)
XRRRQRRKKRX

SEQ ID NO: 9 - protein, 31 aa, Artificial
  Description of sequence L-TAT-IB1 (s) (see Table 1)
GRKKRRQRRRPPRPKRPTTLNLFPQVPRSQD

SEQ ID NO: 10 - protein, 29 aa, Artificial
  Description of sequence Peptide L-TAT (generic) (s) (see Table 1)
XRKKRRQRRRXXRPTTLXLXXXXXXXQDX

SEQ ID NO: 11 - protein, 31 aa, Artificial
  Description of sequence Peptid D-TAT-IB1 (s) (see Table 1)
DQSRPVQPFLNLTTPRKPRPPRRRQRRKKRG

SEQ ID NO: 12 - protein, 29 aa, Artificial
  Description of sequence Peptid D-TAT (generic) (s) (see Table 1)
XDQXXXXXXXLXLTTPRXXRRRQRRKKRX

SEQ ID NO: 13 - protein, 29 aa, Artificial
  Description of sequence peptide IB1-long (see Table 1)
PGTGCGDTYRPKRPTTLNLFPQVPRSQDT

SEQ ID NO: 14 - protein, 27 aa, Artificial
  Description of sequence Peptide IB2-long (see Table 1)
IPSPSVEEPHKHRPTTLRLTTLGAQDS

SEQ ID NO: 15 - protein, 29 aa, Artificial
  Description of sequence Peptide derived from c-Jun (see Table 1)
GAYGYSNPKILKQSMTLNLADPVGNLKPH

SEQ ID NO: 16 - protein, 29 aa, Artificial
  Description of sequence Peptide derived from ATF2 (see Table 1)
TNEDHLAVHKHKHEMTLKFGPARNDSVIV

SEQ ID NO: 17 - protein, 23 aa, Artificial
  Description of sequence Peptide L-IB1 (see Table 1)
DTYRPKRPTTLNLFPQVPRSQDT

SEQ ID NO: 18 - protein, 23 aa, Artificial
  Description of sequence Peptide D-IB1 (see Table 1)
TDQSRPVQPFLNLTTPRKPRYTD

SEQ ID NO: 19 - protein, 19 aa, Artificial
  Description of sequence Peptide L-IB (generic) (see Table 1)
XRPTTLXLXXXXXXXQDXX

SEQ ID NO: 20 - protein, 19 aa, Artificial
  Description of sequence Peptide D-IB (generic) (see Table 1)
XXDQXXXXXXXLXLTTPRX

SEQ ID NO: 21 - protein, 17 aa, Artificial
  Description of sequence Peptide L-generic-TAT (see Table 1)
XXXXRKKRRQRRRXXXX

SEQ ID NO: 22 - protein, 17 aa, Artificial
  Description of sequence Peptide D-generic-TAT (see Table 1)
XXXXRRRQRRKKRXXXX

SEQ ID NO: 23 - protein, 35 aa, Artificial
  Description of sequence Peptide L-TAT-IB1 (see Table 1)
GRKKRRQRRRPPDTYRPKRPTTLNLFPQVPRSQDT

SEQ ID NO: 24 - protein, 42 aa, Artificial
  Description of sequence Peptide L-TAT IB (generic) (see Table 1)
XXXXXXXRKKRRQRRRXXXXXXXXRPTTLXLXXXXXXXQDXX

SEQ ID NO: 25 - protein, 35 aa, Artificial
  Description of sequence Peptide D-TAT-IB1 (see Table 1)
TDQSRPVQPFLNLTTPRKPRYTDPPRRRQRRKKRG

SEQ ID NO: 26 - protein, 42 aa, Artificial
  Description of sequence Peptide D-TAT IB (generic) (see Table 1)
XXDQXXXXXXXLXLTTPRXXXXXXXXRRRQRRKKRXXXXXXX

SEQ ID NO: 27 - protein, 30 aa, Artificial
  Description of sequence chimeric peptide sequence L-TAT-IB1(s1) (see Table 1)
RKKRRQRRRPPRPKRPTTLNLFPQVPRSQD

SEQ ID NO: 28 - protein, 30 aa, Artificial
  Description of sequence chimeric peptide sequence L-TAT-IB1(s2) (see Table 1)
GRKKRRQRRRXRPKRPTTLNLFPQVPRSQD

SEQ ID NO: 29 - protein, 29 aa, Artificial
  Description of sequence chimeric peptide sequence L-TAT-IB1(s3) (see Table 1)
RKKRRQRRRXRPKRPTTLNLFPQVPRSQD

SEQ ID NO: 30 - protein, 30 aa, Artificial
  Description of sequence chimeric peptide sequence D-TAT-IB1(s1) (see Table 1)
DQSRPVQPFLNLTTPRKPRPPRRRQRRKKR

SEQ ID NO: 31 - protein, 30 aa, Artificial
  Description of sequence chimeric peptide sequence D-TAT-IB1(s2) (see Table 1)
DQSRPVQPFLNLTTPRKPRXRRRQRRKKRG

SEQ ID NO: 32 - protein, 29 aa, Artificial
  Description of sequence chimeric peptide sequence D-TAT-IB1(s3) (see Table 1)
DQSRPVQPFLNLTTPRKPRXRRRQRRKKR

SEQ ID NO: 33 - protein, 13 aa, Artificial
  Description of sequence L-IB1(s1) (see Table 1)
TLNLFPQVPRSQD

SEQ ID NO: 34 - protein, 13 aa, Artificial
  Description of sequence L-IB1(s2) (see Table 1)
TTLNLFPQVPRSQ

SEQ ID NO: 35 - protein, 13 aa, Artificial
  Description of sequence L-IB1(s3) (see Table 1)
PTTLNLFPQVPRS

SEQ ID NO: 36 - protein, 13 aa, Artificial
  Description of sequence L-IB1(s4) (see Table 1)
RPTTLNLFPQVPR

SEQ ID NO: 37 - protein, 13 aa, Artificial
  Description of sequence L-IB1(s5) (see Table 1)
KRPTTLNLFPQVP

SEQ ID NO: 38 - protein, 13 aa, Artificial
  Description of sequence L-IB1(s6) (see Table 1)
PKRPTTLNLFPQV

SEQ ID NO: 39 - protein, 13 aa, Artificial
  Description of sequence L-IB1(s7) (see Table 1)
RPKRPTTLNLFPQ

SEQ ID NO: 40 - protein, 12 aa, Artificial
  Description of sequence L-IB1(s8) (see Table 1)
LNLFPQVPRSQD

SEQ ID NO: 41 - protein, 12 aa, Artificial
  Description of sequence L-IB1(s9) (see Table 1)
TLNLFPQVPRSQ

SEQ ID NO: 42 - protein, 12 aa, Artificial
  Description of sequence L-IB1(s10) (see Table 1)
TTLNLFPQVPRS

SEQ ID NO: 43 - protein, 12 aa, Artificial
  Description of sequence L-IB1(s11) (see Table 1)
PTTLNLFPQVPR

SEQ ID NO: 44 - protein, 12 aa, Artificial
  Description of sequence L-IB1(s12) (see Table 1)
RPTTLNLFPQVP

SEQ ID NO: 45 - protein, 12 aa, Artificial
  Description of sequence L-IB1(s13) (see Table 1)
KRPTTLNLFPQV

SEQ ID NO: 46 - protein, 12 aa, Artificial
  Description of sequence L-IB1(s14) (see Table 1)
PKRPTTLNLFPQ

SEQ ID NO: 47 - protein, 12 aa, Artificial
  Description of sequence L-IB1(s15) (see Table 1)
RPKRPTTLNLFP

SEQ ID NO: 48 - protein, 11 aa, Artificial
  Description of sequence L-IB1(s16) (see Table 1)
NLFPQVPRSQD

SEQ ID NO: 49 - protein, 11 aa, Artificial
  Description of sequence L-IB1(s17) (see Table 1)
LNLFPQVPRSQ

SEQ ID NO: 50 - protein, 11 aa, Artificial
  Description of sequence L-IB1(s18) (see Table 1)
TLNLFPQVPRS

SEQ ID NO: 51 - protein, 11 aa, Artificial
  Description of sequence L-IB1(s19) (see Table 1)
TTLNLFPQVPR

SEQ ID NO: 52 - protein, 11 aa, Artificial
  Description of sequence L-IB1(s20) (see Table 1)
PTTLNLFPQVP

SEQ ID NO: 53 - protein, 11 aa, Artificial
  Description of sequence L-IB1(s21) (see Table 1)
RPTTLNLFPQV

SEQ ID NO: 54 - protein, 11 aa, Artificial
  Description of sequence L-IB1(s22) (see Table 1)
KRPTTLNLFPQ

SEQ ID NO: 55 - protein, 11 aa, Artificial
  Description of sequence L-IB1(s23) (see Table 1)
PKRPTTLNLFP

SEQ ID NO: 56 - protein, 11 aa, Artificial
  Description of sequence L-IB1(s24) (see Table 1)
RPKRPTTLNLF

SEQ ID NO: 57 - protein, 10 aa, Artificial
  Description of sequence L-IB1(s25) (see Table 1)
LFPQVPRSQD

SEQ ID NO: 58 - protein, 10 aa, Artificial
  Description of sequence L-IB1(s26) (see Table 1)
NLFPQVPRSQ

SEQ ID NO: 59 - protein, 10 aa, Artificial
  Description of sequence L-IB1(s27) (see Table 1)
LNLFPQVPRS

SEQ ID NO: 60 - protein, 10 aa, Artificial
  Description of sequence L-IB1(s28) (see Table 1)
TLNLFPQVPR

SEQ ID NO: 61 - protein, 10 aa, Artificial
  Description of sequence L-IB1(s29) (see Table 1)
TTLNLFPQVP

SEQ ID NO: 62 - protein, 10 aa, Artificial
  Description of sequence L-IB1(s30) (see Table 1)
PTTLNLFPQV

SEQ ID NO: 63 - protein, 10 aa, Artificial
  Description of sequence L-IB1(s31) (see Table 1)
RPTTLNLFPQ

SEQ ID NO: 64 - protein, 10 aa, Artificial
  Description of sequence L-IB1(s32) (see Table 1)
KRPTTLNLFP

SEQ ID NO: 65 - protein, 10 aa, Artificial
  Description of sequence L-IB1(s33) (see Table 1)
PKRPTTLNLF

SEQ ID NO: 66 - protein, 10 aa, Artificial
  Description of sequence L-IB1(s34) (see Table 1)
RPKRPTTLNL

SEQ ID NO: 67 - protein, 13 aa, Artificial
  Description of sequence D-IB1(s1) (see Table 1)
QPFLNLTTPRKPR

SEQ ID NO: 68 - protein, 13 aa, Artificial
  Description of sequence D-IB1(s2) (see Table 1)
VQPFLNLTTPRKP

SEQ ID NO: 69 - protein, 13 aa, Artificial
  Description of sequence D-IB1(s3) (see Table 1)
PVQPFLNLTTPRK

SEQ ID NO: 70 - protein, 13 aa, Artificial
  Description of sequence D-IB1(s4) (see Table 1)
RPVQPFLNLTTPR

SEQ ID NO: 71 - protein, 13 aa, Artificial
  Description of sequence D-IB1(s5) (see Table 1)
SRPVQPFLNLTTP

SEQ ID NO: 72 - protein, 13 aa, Artificial
  Description of sequence D-IB1(s6) (see Table 1)
QSRPVQPFLNLTT

SEQ ID NO: 73 - protein, 13 aa, Artificial
  Description of sequence D-IB1(s7) (see Table 1)
DQSRPVQPFLNLT

SEQ ID NO: 74 - protein, 12 aa, Artificial
  Description of sequence D-IB1(s8) (see Table 1)
PFLNLTTPRKPR

SEQ ID NO: 75 - protein, 12 aa, Artificial
  Description of sequence D-IB1(s9) (see Table 1)
QPFLNLTTPRKP

SEQ ID NO: 76 - protein, 12 aa, Artificial
  Description of sequence D-IB1(s10) (see Table 1)
VQPFLNLTTPRK

SEQ ID NO: 77 - protein, 12 aa, Artificial
  Description of sequence D-IB1(s11) (see Table 1)
PVQPFLNLTTPR

SEQ ID NO: 78 - protein, 12 aa, Artificial
  Description of sequence D-IB1(s12) (see Table 1)
RPVQPFLNLTTP

SEQ ID NO: 79 - protein, 12 aa, Artificial
  Description of sequence D-IB1(s13) (see Table 1)
SRPVQPFLNLTT

SEQ ID NO: 80 - protein, 12 aa, Artificial
  Description of sequence D-IB1(s14) (see Table 1)
QSRPVQPFLNLT

SEQ ID NO: 81 - protein, 12 aa, Artificial
  Description of sequence D-IB1(s15) (see Table 1)
DQSRPVQPFLNL

SEQ ID NO: 82 - protein, 11 aa, Artificial
  Description of sequence D-IB1(s16) (see Table 1)
FLNLTTPRKPR

SEQ ID NO: 83 - protein, 11 aa, Artificial
  Description of sequence D-IB1(s17) (see Table 1)
PFLNLTTPRKP

SEQ ID NO: 84 - protein, 11 aa, Artificial
  Description of sequence D-IB1(s18) (see Table 1)
QPFLNLTTPRK

SEQ ID NO: 85 - protein, 11 aa, Artificial
  Description of sequence D-IB1(s19) (see Table 1)
VQPFLNLTTPR

SEQ ID NO: 86 - protein, 11 aa, Artificial
  Description of sequence D-IB1(s20) (see Table 1)
PVQPFLNLTTP

SEQ ID NO: 87 - protein, 11 aa, Artificial
  Description of sequence D-IB1(s21) (see Table 1)
RPVQPFLNLTT

SEQ ID NO: 88 - protein, 11 aa, Artificial
  Description of sequence D-IB1(s22) (see Table 1)
SRPVQPFLNLT

SEQ ID NO: 89 - protein, 11 aa, Artificial
  Description of sequence D-IB1(s23) (see Table 1)
QSRPVQPFLNL

SEQ ID NO: 90 - protein, 11 aa, Artificial
  Description of sequence D-IB1(s24) (see Table 1)
DQSRPVQPFLN

SEQ ID NO: 91 - protein, 10 aa, Artificial
  Description of sequence D-IB1(s25) (see Table 1)
DQSRPVQPFL

SEQ ID NO: 92 - protein, 10 aa, Artificial
  Description of sequence D-IB1(s26) (see Table 1)
QSRPVQPFLN

SEQ ID NO: 93 - protein, 10 aa, Artificial
  Description of sequence D-IB1(s27) (see Table 1)
SRPVQPFLNL

SEQ ID NO: 94 - protein, 10 aa, Artificial
  Description of sequence D-IB1(s28) (see Table 1)
RPVQPFLNLT

SEQ ID NO: 95 - protein, 10 aa, Artificial
  Description of sequence D-IB1(s29) (see Table 1)
PVQPFLNLTT

SEQ ID NO: 96 - protein, 10 aa, Artificial
  Description of sequence D-IB1(s30) (see Table 1)
VQPFLNLTTP

SEQ ID NO: 97 - protein, 10 aa, Artificial
  Description of sequence D-IB1(s31) (see Table 1)
QPFLNLTTPR

SEQ ID NO: 98 - protein, 10 aa, Artificial
  Description of sequence D-IB1(s32) (see Table 1)
PFLNLTTPRK

SEQ ID NO: 99 - protein, 10 aa, Artificial
  Description of sequence D-IB1(s33) (see Table 1)
FLNLTTPRKP

SEQ ID NO: 100 - protein, 10 aa, Artificial
  Description of sequence D-IB1(s34) (see Table 1)
LNLTTPRKPR

SEQ ID NO: 101 - DNA, 20 bp, Artificial
  Primer specific for GAPDH (Forward)
atgcccccatgtttgtgatg

SEQ ID NO: 102 - DNA, 2953 bp, Rattus norvegicus
ccgccccagctcagtccgaaccccgcggcggcggcggcctcctccacacgcctccacctccgccgccgccgccgccgccgccgcctcccgcgccgctctccgcccggatggccaggctgagcccgggaatggcggagcgagagagcggcctgagcgggggtgccgcgtccccaccggccgcttccccattcctgggactgcacatcgcgtcgcctcccaatttcaggctcacccatgatatcagcctggaggagtttgaggatgaagacctttcggagatcactgatgagtgtggcatcagcctgcagtgcaaagacaccttgtctctccggcccccgcgcgccgggctactgtctgcgggtagcagcggtagcgcggggagccggctgcaggcggagatgctgcagatggacctgatcgacgcggcaagtgacactccgggcgccgaggacgacgaagaggacgacgacgagctcgctgcccaacggccaggagtggggccttccaaagccgagtctggccaggagccggcgtctcgcagccagggtcagggccagggccccggcacaggctgcggagacacctaccggcccaagaggcctaccacgctcaaccttttcccgcaggtgccgcggtctcaggacacgctgaataataactctttaggcaaaaagcacagttggcaggaccgtgtgtctcgatcatcctcccctctgaagacaggggagcagacgcctccacatgaacatatctgcctgagtgatgagctgccgccccagggcagtcctgttcccacccaggatcgtggcacttccaccgacagcccttgtcgccgtactgcagccacccagatggcacctccaagtggtccccctgccactgcacctggtggccggggccactcccatcgagatcggtccatatcagcagatgtgcggctcgaggcgactgaggagatctacctgaccccagtgcagaggcccccagaccctgcagaacccacctccaccttcttgccacccactgagagccggatgtctgtcagctcggatcctgaccctgccgcttactctgtaactgcagggcgaccgcacccttccatcagtgaagaggatgagggcttcgactgtctgtcatccccagagcaagctgagccaccaggtggagggtggcggggaagcctcggggagccaccaccgcctccacgggcctcactgagctcggacaccagcgcactgtcctacgactctgtcaagtacacactggtggtggatgagcatgcccagcttgagttggtgagcctgcggccatgttttggagattacagtgacgaaagcgactctgccactgtctatgacaactgtgcctctgcctcctcgccctacgagtcagccattggtgaggaatatgaggaggcccctcaaccccggcctcccacctgcctgtcagaggactccacaccggatgagcctgacgtccacttctctaagaagtttctgaatgtcttcatgagtggccgctctcgttcctccagtgccgagtcctttgggctgttctcctgtgtcatcaatggggaggagcatgagcaaacccatcgggctatattcaggtttgtgcctcggcatgaagatgaacttgagctggaagtggacgaccctctgctggtggagctgcaggcagaagactattggtatgaggcctataacatgcgcactggagcccgtggtgtctttcctgcctactatgccattgaggtcaccaaggagcctgagcacatggcagcccttgccaaaaacagcgactggattgaccagttccgggtgaagttcctgggctctgtccaggttccttatcacaagggcaatgatgtcctctgtgctgctatgcaaaagatcgccaccacccgccggctcaccgtgcactttaacccgccctccagctgtgtccttgaaatcagcgttaggggtgtcaagataggtgtcaaagctgatgaagctcaggaggccaagggaaataaatgtagccactttttccagctaaaaaacatctctttctgtgggtaccatccaaagaacaacaagtactttgggtttatcactaagcaccctgctgaccaccggtttgcctgccatgtctttgtgtctgaagattccaccaaagccctggcagagtctgtggggcgtgcatttcagcagttctacaagcaatttgtggaatatacctgtcctacagaagatatctacttggagtagcagcaacccccctctctgcagcccctcagccccaggccagtactaggacagctgactgctgacaggatgttgtactgccacgagagaatgggggagtgagggctgttggggtcggggggcaggggtttggggagaggcagatgcagtttattgtaatatatggggttagattaatctatggaggacagtacaggctctctcggggctggggaagggcagggctggggtgggggtcaggcatctggccacaaaggggtcccctagggacagaggcgctgcaccatcctgggcttgtttcatactagaggccctggctttctggctcttgggtcctgccttgacaaagcccagccacctggaagtgtcaccttcccttgtccacctcacccagtgccctgagctcatgctgagcccaagcacctccgaaggactttccagtaaggaaatggcaacatgtgacagtgagaccctgttctcatctgtggggctccggcagctccgacccccagcctggccagcacgctgaccctggcaagcttgtgtgttcaaagaaggagagggccacagcaagccctgcctgccagggaaggttccctctcagctggccccagccaactggtcactgtcttgtcacctggctactactattaaagtgccatttcttgtctgaaaaaaaaaaaaaaaaaaaaaaaaaactcgag

SEQ ID NO: 103 - protein, 714 aa, Rattus norvegicus
MARLSPGMAERESGLSGGAASPPAASPFLGLHIASPPNFRLTHDISLEEFEDEDLSEITDECGISLQCKDTLSLRPPRAGLLSAGSSGSAGSRLQAEMLQMDLIDAASDTPGAEDDEEDDDELAAQRPGVGPSKAESGQEPASRSQGQGQGPGTGCGDTYRPKRPTTLNLFPQVPRSQDTLNNNSLGKKHSWQDRVSRSSSPLKTGEQTPPHEHICLSDELPPQGSPVPTQDRGTSTDSPCRRTAATQMAPPSGPPATAPGGRGHSHRDRSISADVRLEATEEIYLTPVQRPPDPAEPTSTFLPPTESRMSVSSDPDPAAYSVTAGRPHPSISEEDEGFDCLSSPEQAEPPGGGWRGSLGEPPPPPRASLSSDTSALSYDSVKYTLVVDEHAQLELVSLRPCFGDYSDESDSATVYDNCASASSPYESAIGEEYEEAPQPRPPTCLSEDSTPDEPDVHFSKKFLNVFMSGRSRSSSAESFGLFSCVINGEEHEQTHRAIFRFVPRHEDELELEVDDPLLVELQAEDYWYEAYNMRTGARGVFPAYYAIEVTKEPEHMAALAKNSDWIDQFRVKFLGSVQVPYHKGNDVLCAAMQKIATTRRLTVHFNPPSSCVLEISVRGVKIGVKADEAQEAKGNKCSHFFQLKNISFCGYHPKNNKYFGFITKHPADHRFACHVFVSEDSTKALAESVGRAFQQFYKQFVEYTCPTEDIYLE

SEQ ID NO: 104 - protein, 711 aa, Homo sapiens
MAERESGGLGGGAASPPAASPFLGLHIASPPNFRLTHDISLEEFEDEDLSEITDECGISLQCKDTLSLRPPRAGLLSAGGGGAGSRLQAEMLQMDLIDATGDTPGAEDDEEDDDEERAARRPGAGPPKAESGQEPASRGQGQSQGQSQGPGSGDTYRPKRPTTLNLFPQVPRSQDTLNNNSLGKKHSWQDRVSRSSSPLKTGEQTPPHEHICLSDELPPQSGPAPTTDRGTSTDSPCRRSTATQMAPPGGPPAAPPGGRGHSHRDRIHYQADVRLEATEEIYLTPVQRPPDAAEPTSAFLPPTESRMSVSSDPDPAAYPSTAGRPHPSISEEEEGFDCLSSPERAEPPGGGWRGSLGEPPPPPRASLSSDTSALSYDSVKYTLVVDEHAQLELVSLRPCFGDYSDESDSATVYDNCASVSSPYESAIGEEYEEAPRPQPPACLSEDSTPDEPDVHFSKKFLNVFMSGRSRSSSAESFGLFSCIINGEEQEQTHRAIFRFVPRHEDELELEVDDPLLVELQAEDYWYEAYNMRTGARGVFPAYYAIEVTKEPEHMAALAKNSDWVDQFRVKFLGSVQVPYHKGNDVLCAAMQKIATTRRLTVHFNPPSSCVLEISVRGVKIGVKADDSQEAKGNKCSHFFQLKNISFCGYHPKNNKYFGFITKHPADHRFACHVFVSEDSTKALAESVGRAFQQFYKQFVEYTCPTEDIYLE

SEQ ID NO: 105 - DNA, 2136 bp, Homo sapiens
atggcggagcgagaaagcggcggcctgggagggggggccgcgtccccgcccgccgcctccccgttcctggggctgcacatcgcttcgcctcccaatttcaggctcacccatgacatcagcctggaggagtttgaggatgaagacctctcggagatcactgatgagtgtggcatcagcttacagtgcaaagacaccctgtccttacggcccccgcgcgccgggctgctctctgcgggcggcggcggcgcggggagccggttgcaggccgagatgctgcagatggacctgatcgacgcgacgggggacactcccggggccgaggacgacgaggaggacgacgacgaggagcgcgcggcccggcggccgggagcggggccgcccaaggccgagtccggccaggagccggcgtcccgcggccagggccagagccaaggccagagccagggcccgggcagcggggacacgtaccggcccaagcggcccaccacgctcaacctctttccgcaggtgccgcggtctcaggacacactgaataataattctctgggcaaaaagcacagttggcaggatcgggtgtctcgatcatcctcacccctgaagacaggggagcagacaccaccgcatgaacacatctgcctgagcgatgagctgcccccccagagcggccccgcccccaccacagatcgaggcacctccaccgacagcccttgccgccgcagcacagccacccagatggcacctccgggtggtccccctgctgccccgcctgggggtcggggccactcgcatcgagaccgaatccactaccaggccgatgtgcgactagaggccactgaggagatctacctgaccccagtgcagaggcccccagacgctgcagagcccacctccgccttcctgccgcccactgagagccggatgtcagtcagctccgatccagaccctgccgcctacccctccacggcagggcggccgcacccctccatcagtgaagaggaagagggcttcgactgcctgtcgtccccagagcgggctgagcccccaggcggagggtggcgggggagcctgggggagccgccgccacctccacgggcctctctgagctcggacaccagcgccctgtcctatgactctgtcaagtacacgctggtggtagatgagcatgcacagctggagctggtgagcctgcggccgtgcttcggagactacagtgacgagagtgactctgccaccgtctatgacaactgtgcctccgtctcctcgccctatgagtcggccatcggagaggaatatgaggaggccccgcggccccagccccctgcctgcctctccgaggactccacgcctgatgaacccgacgtccatttctccaagaaattcctgaacgtcttcatgagtggccgctcccgctcctccagtgctgagtccttcgggctgttctcctgcatcatcaacggggaggagcaggagcagacccaccgggccatattcaggtttgtgcctcgacacgaagacgaacttgagctggaagtggatgaccctctgctagtggagctccaggctgaagactactggtacgaggcctacaacatgcgcactggtgcccggggtgtctttcctgcctattacgccatcgaggtcaccaaggagcccgagcacatggcagccctggccaaaaacagtgactgggtggaccagttccgggtgaagttcctgggctcagtccaggttccctatcacaagggcaatgacgtcctctgtgctgctatgcaaaagattgccaccacccgccggctcaccgtgcactttaacccgccctccagctgtgtcctggagatcagcgtgcggggtgtgaagataggcgtcaaggccgatgactcccaggaggccaaggggaataaatgtagccactttttccagttaaaaaacatctctttctgcggatatcatccaaagaacaacaagtactttgggttcatcaccaagcaccccgccgaccaccggtttgcctgccacgtctttgtgtctgaagactccaccaaagccctggcagagtccgtggggagagcattccagcagttctacaagcagtttgtggagtacacctgccccacagaagatatctacctggagtag